(19) United States
(12) Patent Application Publication
(10) Pub. No.: US 2011/0223597 A1
(43) Pub. Date: Sep. 15, 2011

(54) METHODS RELATING TO OLANZAPINE PHARMACOGENETICS

(75) Inventors: Philip Lazarus, Hummelstown, PA (US); Kathryn Erickson, Hummelstown, PA (US)

(73) Assignee: The Penn State Research Foundation, University Park, PA (US)

(21) Appl. No.: 13/045,171

(22) Filed: Mar. 10, 2011

Related U.S. Application Data
(60) Provisional application No. 61/312,433, filed on Mar. 10, 2010.

(51) Int. Cl. C12Q 1/68; C12Q 1/48; G01N 33/573; C07H 21/00; C07K 16/40

(52) U.S. Cl. 435/6.11; 435/15; 435/7.4; 536/24.33; 536/24.31; 530/389.8

(57) ABSTRACT

There is significant variability in subject clearance, half-life and side-effects from treatment with olanzapine (OLZ) in subjects. Methods for aiding in determining therapeutic efficacy of olanzapine in a subject are provided according to embodiments of the present invention which include identifying in a subject sample whether UDP-glucuronosyltransferase 2B10 (UGT2B10) and/or UDP-glucuronosyltransferase 1A4 (UGT1A4) is “wild-type” or a variant associated with altered glucuronidation of an olanzapine metabolite compared to wild-type.

### REFERENCE TO RELATED APPLICATION

This application claims priority from U.S. Provisional Patent Application Ser. No. 61/312,433, filed Mar. 10, 2010, the entire content of which is incorporated herein by reference.

### FIELD OF THE INVENTION

Embodiments of the present invention provide methods relating to olanzapine pharmacogenetics in a subject. Aspects of the present invention include methods and kits for aiding in determining therapeutic efficacy of olanzapine treatment in a subject in need thereof.

### BACKGROUND OF THE INVENTION

Olanzapine (OLZ) is an atypical antipsychotic used for the treatment of schizophrenia; bipolar disorder; and other conditions including, but not limited to treatment of psychosis associated with Alzheimers dementia; cluster headache; and chemotherapy-induced nausea. There is significant variability in subject clearance, half-life and side-effects from treatment with OLZ. It is desirable to have as much information as possible to aid in determination of whether OLZ is or will be therapeutically effective in a subject.

### SUMMARY OF THE INVENTION

Methods for aiding in determining therapeutic efficacy of olanzapine in a subject are provided according to embodiments of the present invention which include identifying in a subject sample the amino acid encoded by genomic DNA or mRNA encoding UDP-glucuronosyltransferase 2B10 (UGT2B10) at amino acid position 67 of UDP-glucuronosyltransferase 2B10, wherein Asp at position 67 of the UDP-glucuronosyltransferase 2B10 is correlated with increased glucuronidation of olanzapine compared to Tyr at position 67. A reference amino acid sequence of human UGT2B10 is shown herein as SEQ ID NO. 1 which shows the “wild-type” UGT2B10 sequence having Asp (D) at position 67, a form of the UGT2B10 protein correlated with increased glucuronidation of olanzapine compared to UGT2B10 protein having Tyr at position 67.

Methods for aiding in determining therapeutic efficacy of olanzapine in a subject are provided according to embodiments of the present invention which include assaying a subject sample using PCR to identify the amino acid encoded by genomic DNA or mRNA encoding UDP-glucuronosyltransferase 2B10 (UGT2B10) at amino acid position 67 of UDP-glucuronosyltransferase 2B10, wherein Asp at position 67 of the UDP-glucuronosyltransferase 2B10 is correlated with increased glucuronidation of olanzapine compared to Tyr at position 67.

Methods for aiding in determining therapeutic efficacy of olanzapine in a subject are provided according to embodiments of the present invention which identifying in a subject sample the amino acid encoded by genomic DNA or mRNA encoding UDP-glucuronosyltransferase 1A4 (UGT1A4) at amino acid position 24 of UDP-glucuronosyltransferase 1A4, wherein Thr(T) at position 24 or Pro(P) at position 24 is correlated with altered glucuronidation of olanzapine compared to UGT1A4 proteins having Pro (P) at position 24.

Methods for aiding in determining therapeutic efficacy of olanzapine in a subject are provided according to embodiments of the present invention which identifying in a subject sample the amino acid encoded by genomic DNA or mRNA encoding UDP-glucuronosyltransferase 1A4 (UGT1A4) at amino acid position 48 of UDP-glucuronosyltransferase 1A4, wherein Val(V) at position 48 is correlated with altered glucuronidation of olanzapine compared to UGT1A4 proteins having Leu(L) at position 48.

Methods for aiding in determining therapeutic efficacy of olanzapine in a subject are provided according to embodiments of the present invention which identifying in a subject sample the amino acid encoded by genomic DNA or mRNA encoding UDP-glucuronosyltransferase 1A4 (UGT1A4) at amino acid positions 24 and 48 of UDP-glucuronosyltransferase 1A4, wherein Thr(T) at position 24 and Leu (L) at position 48 of the UDP-glucuronosyltransferase 1A4 or Pro(P) at position 24 and Val (V) at position 48 of the UDP-glucuronosyltransferase 1A4 is correlated with altered glucuronidation of olanzapine compared to UGT1A4 proteins having Pro (P) at position 24 and Leu (L) at position 48.

A reference amino acid sequence of human UGT1A4 is shown herein as SEQ ID NO. 3 which shows the “wild-type” UGT1A4 sequence having Pro (P) at position 24 and Leu (L) at position 48, a form of the UGT1A4 protein correlated with normal (reference) glucuronidation of olanzapine.

Further shown is a related UGT1A4 amino acid sequence, SEQ ID NO. 4, which lacks three N-terminal amino acids compared to the UGT1A4 of SEQ ID NO. 3 such that the protein has the Pro(P) at position 21 and Val (V) at position 45, corresponding to Pro(P) at position 24 and Val (V) at position 48 of SEQ ID NO. 3. While wild-type and mutant UGT1A4 amino acid sequence are mentioned herein with respect to amino acids 24 and 48 corresponding to their positions in SEQ ID NO. 3, the same numbering is also used to refer to the analogous amino acids in SEQ ID NO. 4, actually present at positions 21 and 45. One of skill in the art will recognize the positions referred to by these names in the analogous sequence.

Methods for aiding in determining therapeutic efficacy of olanzapine in a subject are provided according to embodiments of the present invention which include assaying a subject sample using PCR to identify in the amino acid encoded by genomic DNA or mRNA encoding UDP-glucuronosyltransferase 1A4 (UGT1A4) at amino acid positions 24 and/or 48 of UDP-glucuronosyltransferase 1A4, wherein Thr(T) at position 24 is correlated with altered glucuronidation of olanzapine compared to UGT1A4 proteins having Pro (P) at position 24; and wherein Val (V) at position 48 of the UDP-glucuronosyltransferase 1A4 is correlated with altered glucuronidation of olanzapine compared to UGT1A4 proteins having Leu (L) at position 48.

Methods for aiding in determining therapeutic efficacy of olanzapine in a subject, are provided according to embodiments of the present invention which include identifying in a subject sample the UDP-glucuronosyltransferase 2B10 as wild-type or D67Y variant, wherein wild-type UDP-glucuronosyltransferase 2B10 is correlated with increased glucuronidation of olanzapine compared to D67Y variant UDP-glucuronosyltransferase 2B10. Methods of identifying the UDP-glucuronosyltransferase 2B10 as wild-type or D67Y variant can include, but are not limited to, polymerase chain reaction (PCR), real-time PCR, immunoassay, mass spectrometry and/or chromatography.

Methods for aiding in determining therapeutic efficacy of olanzapine in a subject, are provided according to embodiments of the present invention which include identifying in a subject sample the UDP-glucuronosyltransferase 2B10 as wild-type or D67Y variant, wherein wild-type UDP-glucuronosyltransferase 2B10 is correlated with increased glucuronidation of olanzapine compared to D67Y variant UDP-glucuronosyltransferase 2B10 and optionally further include measuring expression and/or function of UDP-glucuronosyltransferase 1A4, wherein combined expression and/or function of UDP-glucuronosyltransferase 2B10 and UDP-glucuronosyltransferase 1A4 is correlated with therapeutic efficacy of olanzapine in the subject.

Methods for aiding in determining therapeutic efficacy of olanzapine in a subject are provided which include identifying the amino acid encoded by genomic DNA or mRNA at amino acid position 24 and 48 of UDP-glucuronosyltransferase 1A4.

Methods for aiding in determining therapeutic efficacy of olanzapine in a subject are provided which include obtaining a sample comprising UDP-glucuronosyltransferase 1A4 from the subject; and identifying the UDP-glucuronosyltransferase 1A4 as wild-type or a variant by identification of the amino acid at amino acid position 24 and/or 48 of UDP-glucuronosyltransferase 1A4.

Kits for aiding in determining therapeutic efficacy of olanzapine in a subject are provided according to embodiments of the present invention including at least one reagent for detection of a UDP-glucuronosyltransferase 2B10 nucleic acid or protein and/or at least one reagent for detection of a UDP-glucuronosyltransferase 1A4 nucleic acid or protein.

### BRIEF DESCRIPTION OF THE DRAWINGS

FIG. 1 shows the enzymes involved in OLZ metabolism and the relative urinary contribution of each pathway;

FIG. 2A is a trace of UPLC analysis showing results of glucuronidation assays performed using 12.5 μg HLM protein+vehicle (1% DMSO), and incubated at 37° C. for 2 h with 4 mM UDP-glucuronic acid prior to analysis by UPLC;

FIG. 2B is a trace of UPLC analysis showing results of glucuronidation assays performed using 12.5 μg UGT-null HEK 293 cell homogenate protein and 300 μM OLZ, and incubated at 37° C. for 2 h with 4 mM UDP-glucuronic acid prior to analysis by UPLC;

FIG. 2C is a trace of UPLC analysis showing results of glucuronidation assays performed using 12.5 μg HLM protein and 300 μM OLZ, and incubated at 37° C. for 2 h with 4 mM UDP-glucuronic acid prior to analysis by UPLC;

FIG. 2D is a trace of UPLC analysis showing results of glucuronidation assays performed using 12.5 μg ELM protein, 300 μM OLZ and 1,000 U of β-glucuronidase, and incubated at 37° C. for 2 h with 4 mM UDP-glucuronic acid prior to analysis by UPLC;

FIG. 2E is a trace showing results of MS analysis for OLZ-glucuronide products of OLZ incubated with HLM, peaks 1 and 2, peak 1, OLZ-10-N-glucuronide isomer 1; peak 2, OLZ-10-N-glucuronide isomer 2, AU, absorbance;

FIG. 2F is a trace showing results of MS analysis for OLZ-glucuronide products of OLZ incubated with HLM, peak 3, peak 3, OLZ-4′-N-glucuronide; peak 4, OLZ; peak 5, UDP-glucuronic acid, AU, absorbance;

FIG. 3A is a representative kinetic plot of glucuronidation rate for OLZ-10-N-glucuronide isomer 1 versus substrate concentration for wild-type UGTs 1A4 (UGT1A4^{24Pro48Leu});

FIG. 3B is a representative kinetic plot of glucuronidation rate for OLZ-10-N-glucuronide isomer 2 versus substrate concentration for wild-type UGTs 1A4 (UGT1A4^{24Pro48Leu});

FIG. 3C Is a representative kinetic plot of glucuronidation rate for OLZ-4′glucuronide versus substrate concentration for wild-type UGTs 1A4 (UGT1A4^{24Pro48Leu});

FIG. 3D is a representative kinetic plot of glucuronidation rate for OLZ-10-N-glucuronide isomer 1 versus substrate concentration for 2B10 (UGT2B10^{67Asp})

FIG. 3E is a representative kinetic plot of glucuronidation rate for OLZ-10-N-glucuronide isomer 2 versus substrate concentration for 2B10 (UGT2B10^{67Asp});

FIG. 3F is a representative kinetic plot of glucuronidation rate for OLZ-4′-glucuronide isomer 1 versus substrate concentration for 2B10 (UGT2B10^{67Asp});

FIG. 3G is a representative kinetic plot of glucuronidation rate for OLZ-10-N-glucuronide isomer 1 versus substrate concentration for HLMs with wild-type UGTs 1A4 and 2B10;

FIG. 3H is a representative kinetic plot of glucuronidation rate for OLZ-10-N-glucuronide isomer 2 versus substrate concentration for HLMs with wild-type UGTs 1A4 and 2B10;

FIG. 3I Is a representative kinetic plot of glucuronidation rate for OLZ-4′-glucuronide versus substrate concentration for HLMs with wild-type UGTs 1A4 and 2B10;

FIG. 4A is a graph showing HLM activity for OLZ-10-N-glucuronide isomer 1 stratified by UGT1A4 genotype;

FIG. 4B is a graph showing HLM activity for OLZ-10-N-glucuronide isomer 2 stratified by UGT1A4 genotype;

FIG. 4C is a graph showing HLM activity for OLZ-4′-N-glucuronide stratified by UGT1A4 genotype;

FIG. 4D is a graph showing HLM activity for all OLZ-glucuronide products stratified by UGT1A4 genotype;

FIG. 4E is a graph showing HLM activity for OLZ-10-N-glucuronide isomer 1 stratified by UGT2B10 genotype;

FIG. 4F is a graph showing HLM activity for OLZ-10-N-glucuronide isomer 2 stratified by UGT2B10 genotype;

FIG. 4G is a graph showing HLM activity for OLZ-4′-N-glucuronide stratified by UGT2B10 genotype;

FIG. 4H is a graph showing HLM activity for all OLZ-glucuronide products stratified by UGT2B10 genotype;

### DETAILED DESCRIPTION OF THE INVENTION

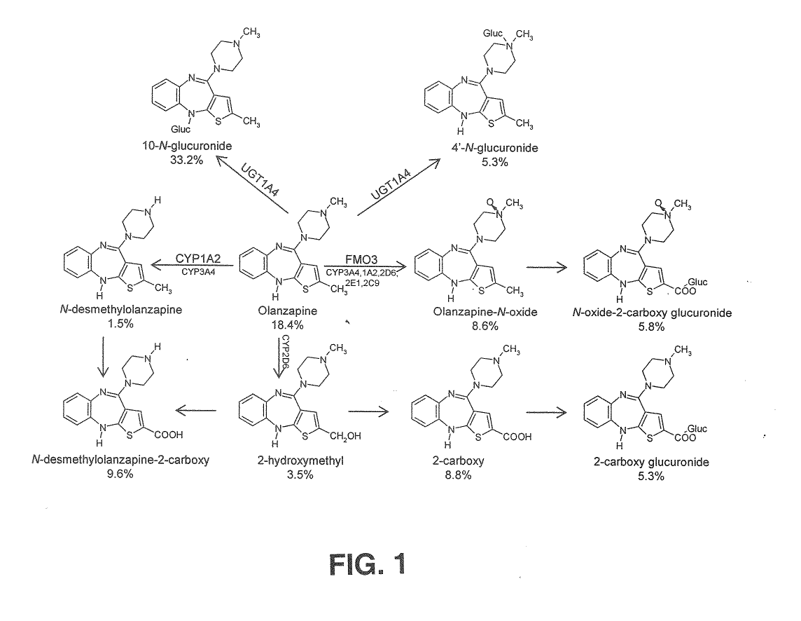

It is an aspect of the present invention that UDP-glucuronosyltransferases UGT2B10 and UGT1A4 are found to have activity to modify at least one metabolite of olanzapine by glucuronidation. Glucuronidated metabolites of olanzapine include olanzapine-4′N-glucuronide and olanzapine-10-N-glucuronide isomers 1 and 2, as shown in FIG. 1.

It is an aspect of the present invention that naturally occurring polymorphisms of UGT2B10 and/or UGT1A4 result in altered levels of glucuronidation of at least one metabolite of olanzapine by UGT2B10 and/or UGT1A4. The altered level of glucuronidation results in altered therapeutic efficacy of olanzapine treatment.

Detection of a UGT2B10 polymorphism in an individual is performed to aid in determination of therapeutic efficacy of olanzapine treatment in the individual. In further embodiments, detection of at least one UGT1A4polymorphism in an individual is performed to aid in determination of therapeutic efficacy of olanzapine treatment in the individual. In still further embodiments, detection of UGT2B10 and UGT1A4 polymorphisms in an individual is performed to aid in determination of therapeutic efficacy of olanzapine treatment in the individual.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808.

The singular terms “a,” “an,” and “the” are not intended to be limiting and include plural referents unless explicitly state or the context clearly indicates otherwise.

The term “therapeutic efficacy” is used to refer to the therapeutic effect of a drug or candidate drug in a condition or disorder in a subject.

Therapeutic effects of olanzapine include reduction of one or more signs and/or symptoms of a psychiatric disease or disorder treated with olanzapine in a subject. Therapeutic effects of olanzapine include reduction of one or more signs and/or symptoms of cluster headache and chemotherapy-induced nausea and/or vomiting in a subject. For example, therapeutic efficacy of olanzapine can be measured by monitoring one or more signs and/or symptoms of schizophrenia, bipolar disorder, psychosis/agitation associated with Alzheimer's dementia, cluster headache and chemotherapy-induced nausea and/or vomiting in a subject.

In particular, it is an aspect of the present invention that a naturally occurring UGT2B10 polymorphism which does not glucuronidate olanzapine has an amino acid sequence in which the amino acid at position 67 is tyrosine. This is in contrast to the reference UGT2B10 amino acid sequence in which the amino acid at position 67 is aspartic acid. A single nucleotide polymorphism responsible for the D67Y variant occurs at nucleotide 199 of a nucleic acid encoding UGT2B10 in which G is replace by T (SNP 199G>T).

Embodiments of the present invention include detecting the D67Y polymorphism in a sample of a subject.

In particular, it is an aspect of the present invention that naturally occurring UGT1A4 variants have altered activity to glucuronidate olanzapine. UGT1A4 variants include UGT1A 24Thr/48Leu and UGT1A4 24Pro/48Val. This is in contrast to the reference UGT1A4 amino acid sequence having 24Pro/48Leu.

Thus, embodiments of the present invention include identifying the amino acids encoded by a UGT1A nucleic acid at amino acid positions 24 and/or 48 of the UGT1A4 protein in a sample of a subject.

Further embodiments of the present invention include detecting the identity of UGT1A4 amino acids at positions 24 and/or 48 of the protein in a sample of a subject.

Characterization of a nucleic acid template to detect a UGT2B10 D67Y and/or UGT1A4 polymorphism in an individual subject is accomplished using any of various techniques, such as polymerase chain reaction (PCR), fluorescent in-situ hybridization (FISH), Southern blot analysis, Northern blot analysis, RNase protection assay, pulsed-field gel electrophoresis (PFGE), direct DNA sequencing and microarray analysis.

Nucleic acid template, such as mRNA, cDNA or genomic DNA template for use in a method described herein is obtained by any of various techniques well-known in the art, exemplified by techniques described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; and D. L. Nelson and M. M. Cox, Lehninger Principles of Biochemistry, 4th Ed., W.H. Freeman & Company, 2004.

The term “polymerase chain reaction” (PCR) encompasses various well-known methods of detecting expression of a nucleic acid including, but not limited to, standard polymerase chain reaction, ligase chain reaction, reverse transcriptase-polymerase chain reaction (RT-PCR) such as quantitative and semi-quantitative RT-PCR, and real-time quantitative RT-PCR. PCR and related methods are well-known molecular biology techniques, disclosed, for example, in U.S. Pat. Nos. 4,683,195 4,683,202, and 4,965,188; and in Bieche et al., International Journal of Cancer, 78:661-666, 1998; Berndt, C. et al., Analytical Biochemistry, 225:252-257, 1995; Gibson, U. et al., Genome Research, 6:995-1001, 1996; Innis et al., PCR Protocols: A Guide to Methods and Applications, Academic Press, 1990; and Piatak et al., BioTechniques, 14:70-81, 1993. PCR primers and methods for their design are well-known in the art, for example, as described in Yuryev, A., PCR Primer Design, Methods in Molecular Biology, Humana Press, 2007.

Amplification products, such as, but not limited to, PCR products, can be analyzed, such as by gel electrophoresis, fluorescent detection, and/or sequencing to detect a mutant and/or wild-type UGT2B10 and/or UGT1A4.

Characterization of a UGT2B10 and/or UGT1A4 peptide or protein to detect a polymorphism in an individual subject is accomplished using any of various techniques, such as binding of a specific binding agent using an immunoassay.

Particular methods of immunoassay are known in the art and illustratively include enzyme-linked immunosorbent assay (ELISA), immunoblot, immunoprecipitation, immunocytochemistry, and radioimmunoassay. Assay methods may be used to obtain qualitative and/or quantitative results. Specific details of suitable assay methods for both qualitative and quantitative assay of a sample are described in standard references, illustratively including E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; Ormerod, M. G., Flow Cytometry: a practical approach, Oxford University Press, 2000; Givan, A. L., Flow Cytometry: first principles, Wiley, New York, 2001; Gorczyca, W., Flow Cytometry in Neoplastic Hematology: morphologic-immunophenotypic correlation, Taylor & Francis, 2006; and J. Sambrook and D. W. Russell, Molecular Cloning: A Laboratory Manual, Cold Spring Harbor Laboratory Press, 3rd Ed., 2001.

Antibodies directed against UGT2B10 and/or UGT1A4 can be polyclonal or monoclonal antibodies. Suitable antibodies also include chimeric antibodies, humanized antibodies, and antigen binding antibody fragments and molecules having antigen binding functionality, such as aptamers. Examples of antibody fragments that can be use in embodiments of inventive assays include Fab fragments, Fab′ fragments, F(ab′)₂ fragments, Fd fragments, Fv fragments, scFv fragments, and domain antibodies (dAb).

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

Aptamers can be used to assay UGT enzymes. The term “aptamer” refers to a peptide and/or nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. Techniques for identification and generation of peptide and nucleic acid aptamers and their use are known in the art as described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

RFLP is a well-known method for detection of a target nucleic acid and is useful in detection of UGT2B10 and/or UGT1A4 polymorphisms. RFLP methods involve digestion of sample nucleic acids with a restriction endonuclease to produce fragments of the nucleic acids. Analysis of the sizes of the fragments allows for detection of polymorphisms due to presence or absence of restriction endonuclease sites in the nucleic acids that differ specifically between nucleic acids of subjects having the polymorphism and subjects without the polymorphism. As is appreciated by those of skill in the art, the choice of restriction endonuclease used depends on the nucleic acids to be analyzed.

Sequencing can be used to detect UGT2B10 and/or UGT1A4 polymorphisms. Sequencing refers to methods of determining the ordered linear sequence of nucleotides or amino acids in a nucleic acid or protein, respectively. Nucleic acid sequencing includes manual and automated techniques using any of various well-known methods including, but not limited to, Maxam-Gilbert sequencing, also known as chemical sequencing; chain termination methods of sequencing, also known as Sanger sequencing; and dye terminator sequencing. Methods of determining the amino acid sequence of a protein or peptide include chemical methods such as Edman degradation.

These and other well-known techniques for detecting protein and nucleic acid polymorphisms are described in detail in standard texts such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering Methods and Protocols, Methods in Molecular Biology, Humana Press, 2003; F. M. Ausubel et al., Eds., Short Protocols in Molecular Biology, Current Protocols, Wiley, 2002; Crowther, J. R., The ELISA Guidebook (Methods in Molecular Biology), Humana Press, 2000; Wild, D., The Immunoassay Handbook, 3rd Edition, Elsevier Science, 2005; J. Sambrook and D. W. Russell, Molecular Cloning: A Laboratory Manual, Cold Spring Harbor Laboratory Press, 3rd Ed., 2001; and Innis et al., PCR Protocols: A Guide to Methods and Applications, Academic Press, 1990.

It is appreciated that due to the degeneracy of the genetic code, one or more nucleotides in a nucleotide sequence encoding UGT1A4 or UGT2B10 may differ from nucleotide sequences shown herein and still encode the UGT1A4 or UGT2B10 proteins of SEQ ID NOs. 1, 3 and 4. Thus, assays for UGT1A4 or UGT2B10 proteins which encode the proteins of SEQ ID NOs. 1, 3 or 4 but which are not identical to nucleotide sequences shown herein are considered to be within the scope of the present invention. Further, as is well-known in the art, variants including one or more conservative amino acid substitutions may occur in a protein without substantial effect on the function of the protein. Thus, one of ordinary skill in the art will appreciate that methods including detection of such UGT1A4 or UGT2B10 variants or nucleic acids encoding such variants are within the scope of the present invention.

In particular embodiments, function of UGT2B10 and/or UGT1A4 to glucuronidate a metabolite of olanzapine is assayed to detect the presence or absence of a UGT2B10 and/or UGT1A4 polymorphism in a sample obtained from a subject.

Any method for detecting function of UGT2B10 and/or UGT1A4 can be used according to embodiments of the present invention.

Optionally, spectrometric analysis is used to assay a sample for a UGT2B10 and/or UGT1A4. For example mass analysis can be used in an assay according to embodiments of the present invention. Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in Li J., et al., Clin Chem., 48(8):1296-304, 2002; Hortin, G. L., Clinical Chemistry 52: 1223-1237, 2006; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

A biological sample assayed according to methods of the present invention is obtained from a subject.

The biological sample is any substance containing or presumed to contain UGT2B10 and/or UGT1A4 nucleic acids or protein such as cells, tissues and fluids including, but not limited to, saliva; blood; semen; mucosal swab, such as buccal swab or nasal swab; hair; tears; stool; secretions of the skin, genitorurinary tract, respiratory tract, intestinal tract; biopsy material, particularly liver biopsy material; and sample of isolated nucleic acids, such as genomic DNA, cDNA and mRNA.

Embodiments of methods of the present invention are described herein with particular reference to samples obtained from a subject, however, such methods are not limited to analysis of samples obtained directly from a subject. For example, genomic DNA template can be obtained from cultured cells, including, but not limited to, cultured cells derived from a human such as cell lines, primary cells or laboratory manipulated cells such as recombinant cells.

A sample from a subject is optionally purified for assay according to a method of the present invention. The term “purified” in the context of a sample refers to separation of UGT2B10 and/or UGT1A4 proteins and/or nucleic acids from at least one other component present in the sample. Sample purification is achieved by techniques illustratively including electrophoretic methods such as gel electrophoresis and 2-D gel electrophoresis; chromatography methods such as HPLC, ion exchange chromatography, affinity chromatography, size exclusion chromatography, thin layer and paper chromatography.

One or more standards can be used for comparison of the standard with UGT2B10 and/or UGT1A4 protein or nucleic acid in a sample.

Assay of UGT2B10 and/or UGT1A4 protein or nucleic acid in a subject sample is optionally compared to assay of an analogue protein or nucleic acid in a control sample. Control samples may be obtained from one or more individual subjects without the described polymorphism of UGT2B10 and/or UGT1A4, also referred to as “wild-type” for example.

Embodiments of kits according to the present invention optionally include one or more components for use in an assay of the present invention such as a UGT2B10 and/or UGT1A4 primer, antibody or aptamer, a liquid such as a buffer and/or solution used in an assay, a container, a detectable label for labeling an antibody or aptamer directly or indirectly, a standard, a negative control and a positive control.

The term “subject” refers to an individual treated and/or to be treated with olanzapine can be any animal, preferably a mammal, such as a human, a non-human primate, cat, dog, sheep, cow, goat, horse, pig, poultry, bird, rabbit and rodent. In preferred embodiments, the subject is human.

In some embodiments, a biological sample is obtained from a subject to aid in determining therapeutic efficacy of olanzapine treatment in the subject. The subject may currently be under treatment with olanzapine or treatment with olanzapine may be contemplated. For example, a subject may have a condition or disorder for which olanzapine treatment is indicated such that the subject is in need thereof.

An individual subject treated and/or to be treated with olanzapine has or is at risk of having a condition or disease for which olanzapine treatment is desirable, such as a psychiatric disease or disorder, chemotherapeutic treatment associated with nausea and/or vomiting and/or cluster headaches.

In other cases, the subject is not under treatment with olanzapine and has no known condition or disorder for which olanzapine treatment is indicated such that the subject is not in need thereof. Methods according to embodiments of the present invention are used in conjunction with a sample from a subject not in need of olanzapine at the time the sample is obtained to aid in determining therapeutic efficacy of olanzapine treatment in the subject in the case such treatment is required in the future. Thus, for example, methods according to the present invention are performed on a sample obtained from the subject at any time, such as prenatally, during infancy, childhood, or any other time to aid in determining therapeutic efficacy of olanzapine treatment in the subject if such treatment is contemplated during the lifetime of the subject.

Methods according to embodiments of the present invention to aid in determining therapeutic efficacy of olanzapine treatment can be performed alone or as part of a panel of biomarkers, such as, but not limited to, biomarkers of disease and/or biomarkers of therapeutic efficacy of various drugs.

Embodiments of inventive compositions and methods are illustrated in the following examples. These examples are provided for illustrative purposes and are not considered limitations on the scope of inventive compositions and methods.

### Examples

### Chemicals and Materials

Olanzapine (OLZ) was purchased from Toronto Chemicals (Toronto, Canada). Alamethicin, β-glucuronidase, bovine serum albumin, anti-calnexin antibody, and lamotrigine (LTG) were purchased from Sigma-Aldrich (St. Louis, Mo.). DMEM, Dulbecco's PBS (minus calcium chloride and magnesium chloride), fetal bovine serum, penicillin-streptomycin, geneticin (G418), Platinum Pfx DNA polymerase, and the pcDNA3.1/V5-His-TOPO mammalian expression vector were all obtained from Invitrogen (Carlsbad, Calif.). The BCA protein assay kit was purchased from Pierce (Rockford, Ill.). The human anti-UGT1A polyclonal antibody and standard were purchased from Gentest (Woburn, Mass.). Anti-β-actin monoclonal antibody was obtained from Sigma. PCR primers were purchased from Integrated DNA Technologies (Coralville, Iowa). All other chemicals were purchased from Fisher Scientific (Waltham, Mass.) unless specified otherwise.

Tissues.

The normal human liver tissue specimens used for these studies are described in detail in Blevins-Primeau, A. et al., Cancer Res. 2009 Mar. 1, 2009; 69(5):1892-900; and Wiener, D. et al., Drug Metab Dispos. 2004 January; 32(1):72-9. Normal adjacent liver specimens were obtained from patients and quick-frozen at −80° C. within 2 h post surgery. ELM were prepared through differential centrifugation as described in Coughtrie, M. et al., Anal Biochem. 1986 November; 159(1):198-205 and stored (10-20 mg microsomal protein/mL) at −80° C. Microsomal protein concentrations were measured using the BCA assay.

Cell Lines.

The cell lines over-expressing the UGT1A and UGT2B isoforms used in this study are described in Chen, G. at al., Pharmacogenetics and Genomics 2008; 18(3):181-91; and Lazarus, P. et al., Ann N Y Acad Sci. 2009 February; 1155:99-111. All UGT over-expressing cell lines were grown in DMEM to 80% confluence before preparing cell homogenates by resuspending pelleted cells in Tris-buffered saline (25 mM Tris base, 138 mM NaCl, and 2.7 mM KCl, pH 7.4) and subjecting them to three rounds of freeze-thaw before gentle homogenization. Total homogenate protein concentrations were measured using the BCA protein assay. Homogenates were stored at −80° C. in 200-μL aliquots to minimize freeze-thaws.

Western Blot Analysis.

UGT1A4 protein levels were determined by Western blot analysis for variant UGT1A4-overexpressing cell lines as described in Sun, D. et al., Breast Cancer Res. 2006; 8(4):R50; and Lazarus, P. et al., Ann N Y Acad Sci. 2009 February; 1155:99-111. UGT1A4 protein levels were quantified against 100-250 ng of human UGT1A protein (Gentest) by densitometric analysis of X-ray film exposures (5 s to 2 min exposures) of Western blots using a GS-800 densitometer with Quantity One software (Bio-Rad, Hercules, Calif.). Quantification was made relative to the levels of calnexin observed in each lane. Antibodies were used at a 1:5000 dilution. Relative UGT1A4 protein levels were expressed as the mean of three independent experiments and all activity assays were normalized relative to UGT1A4 expression in the respective UGT1A4-over-expressing cell line. A similar analysis was not performed for UGT2B10-over-expressing cell lines because an antibody antigenic towards UGT2B10 is not presently available.

Glucuronidation Assays.

Homogenates or HLM were incubated with alamethicin (50 μg/mg protein) for 15 min on ice similar to that described in Sun, D. et al., Breast Cancer Res. 2006; 8(4):R50; and Chen, G. et al., Cancer Research. 2007 Oct. 1, 2007; 67(19):9024-9. Glucuronidation assays were performed in 50 mmol/L Tris buffer (pH 7.5), 10 mmol/L MgCl₂, 4 mmol/L UDP-glucuronic acid (UDPGA), and 7.8 μmol/L to 4 mmol/L of substrate at 37° C. in a water bath for 2 h. HLM (20 μg of protein) or human UGT-over-expressing cell homogenate (50 μg of protein) were screened for glucuronidation activity against OLZ using 50 uM of OLZ in a 10 or 50 μL reaction, respectively. Kinetic assays were performed in 50 μL reactions using a range of 9.4-2000 μM of OLZ, and 50 μg and 1.5 mg of UGT1A4- and UGT2B10-over-expressing cell homogenate protein, respectively, or 12.5 μg of ELM protein. To test the relative contribution of UGT1A4 in overall OLZ glucuronidation, five HLM exhibiting the UGT1A4(*1/*1)/UGT2B10 (*1/*1) genotype were randomly selected and co-treated with 300 μM OLZ and 2000 μM of the UGT1A4 inhibitor LTG. Reactions were terminated by the addition of the same volume of cold acetonitrile as the initial reaction volume. Reactions were centrifuged at 13,000 g for 10 min at 4° C. and supernatants were collected. Glucuronidation assays (5 μL) were analyzed for OLZ glucuronide formation using a Waters ACQUITY ultra pressure liquid chromatography (UPLC) system (Milford, Mass.) as described in Blevins-Primeau, A. et al., Cancer Res. 2009 Mar. 1, 2009; 69(5):1892-900; and Gallagher, C. et al., Cancer Epidemiol Biomarkers Prey. 2007 Apr. 1, 2007; 16(4):823-8 using a 100×2.1 mm i.d. Acquity UPLC Beth C18 column with 1.7 μm particles (Waters) and a 0.2 μm prefilter installed before the column. Elution consisted of a gradient elution starting with 87.5% buffer B (100% acetonitrile) and 17.5% buffer A [5 mmol/L ammonium acetate (pH 6.0)] for 5 min, a linear gradient to 90% buffer B over 1 min and held for 2 min, then a linear gradient back to initial conditions and held for 2 min for a total run time of 10 min. The flow rate was maintained at 0.5 mL/min. The amount of glucuronide formed was determined based on the ratio of OLZ-glucuronide versus unconjugated OLZ after calculating the area under the curve for the OLZ and OLZ-glucuronide peaks using the known amount of OLZ added to each reaction as the reference. OLZ-glucuronide was confirmed by sensitivity to treatment with 1,000 U β-glucuronidase at 37° C. for 12-16 h as described in Wiener, D. et al., Drug Metab Dispos. 2004 January; 32(1):72-9, by treatment with 3 N HCl at 50° C. for 24 h as described in Kassahun, K. et al., Drug Metab Dispos. 1997 Jan. 1, 1997; 25(0:81-93, and by mass spectrometry as described herein. As controls, glucuronidation assays were performed using HLM as a positive control for glucuronidation activity and untransfected HEK293 cell homogenate protein as a negative control for glucuronidation activity. Four independent experiments were performed for kinetic analysis of UGT-over-expressing cell homogenates, with all assays within each experiment performed in duplicate; two independent experiments were performed for rate determination assays and three for kinetic analysis for HLM specimens.

Mass Spectrometry and Ultra Pressure Liquid Chromatography (UPLC).

Triple-quadrupole tandem mass spectrometric detection was performed using an ACQUITY SQD (Waters Corp.) with electrospray ionization interface and an UPLC system consisting of a binary gradient pump, an auto sampler (4° C.), and a column oven (40° C.). UPLC was operated under the same conditions as described herein for glucuronidation assays. Peaks were detected at 270 nm wavelength. The mass spectrometer operated in positive mode was set up to scan the daughter ion of m/z 312.43. The optimized mass spectrometry parameters used were as follows: capillary voltage, 0.57 kV; cone voltage, 30 V; collision energy, 15 V; source temperature, 450° C.; and desolvation temperature, 140° C. Nitrogen was used as the desolvation and cone gas with a flow rate of 760 L/h. Argon was used as the collision gas at a flow rate of 0.1 mL/min. Data acquisition and analysis were performed using the MassLynx NT 4.1 software with QuanLynx program (Waters Corp.).

cDNA Synthesis and Real-Time Expression Assays.

To analyze UGT1A4 and UGT2B10 expression levels in cell lines, real-time PCR was performed using the TaqMan Gene Expression Assay kit from Applied Biosystems [ID: Hs01655285_s1 for UGT1A4, Hs02556282_s1 for UGT2B10 and Hs99999904_m1 for peptidylprolyl isomerase A (cyclophilin A; PPIA) as the housekeeping gene control]. Total RNA was isolated using RNAeasy mini kit (Qiagen). 5 μg of total RNA was reverse-transcribed in a final volume of 20 μL containing 1× reverse transcription-PCR buffer, 0.5 mmol/L of each deoxynucleotide triphosphate, 40 units of recombinant RNase inhibitor, 200 units of SuperScript II reverse transcriptase, and 1 μL of oligo (dT) 12-18 (500 μg/mL) (Invitrogen). Samples were incubated at 42° C. for 60 min, and reverse transcriptase was inactivated by heating at 65° C. for 5 min and cooling to 4° C. Real-time PCR was performed in a volume of 10 μL containing equal amounts of cDNA (20 ng) from each sample. PCRs were carried out in 384-well thin-well PCR plates covered with optically clear sealing film (Applied Biosystems). Amplification, detection, and data analysis were performed using the ABI 7900HT sequence detection system and SDS 2.2.2 software using standard settings provided by ABI (Applied Biosystems). Results were expressed using the comparative threshold method following the recommendations of the manufacturer (Applied Biosystems). The threshold cycle number (CT) value for each was normalized against PPIA and calculated as dCT=CTUGT−CTPPIA. Relative mRNA expression was expressed as fold of UGT versus reference: F=2(−dCT).

UGT Genotyping.

Genotyping for UGT1A4 and UGT2B10 has been previously described for all of the ELM specimens described in this study in Blevins-Primeau, A. et al., Cancer Res. 2009 Mar. 1, 2009; 69(5):1892-900; Chen, G. et al., Pharmacogenetics and Genomics 2008; 18(3):18′-91; and Chen, G. et al., Drug Metab Dispos. 2008 May 1, 2008; 36(5):824-30. Briefly, UGT1A4 genotypes were determined by direct sequencing of PCR-amplified PCR products from liver genomic DNA spanning both codons 24 and 48 for UGT1A4. The same primers were used for both PCR amplification and sequencing of UGT1A4: sense, 5′-GGCTTCTGCTGAGATGGCCAG-3′, (SEQ ID NO. 6) and antisense, 5′-CCTTGAGTGTAGCCCAGCGT-3′, (SEQ ID NO.7), corresponding to nucleotides located −13 to +8 and +277 to +306, respectively, relative to the UGT1A4 translation start site (Genbank accession No. NM_{—}007120). Sequencing was performed using an ABI 3130 Capillary Sequencer at the Functional Genomics Core Facility at the Penn State College of Medicine.

Restriction fragment length polymorphism (RFLP) analysis of the UGT2BIO codon 67 polymorphism (SNP 199G>T) was performed to identify individuals with the UGT2BIO codon 67 polymorphism as described previously (Chen, G. et al., Pharmacogenetics and Genomics 2008; 18(3):181-91). PCR amplification was performed on genomic DNA using a sense primer 5′-AAGGATGGCTCTGAAATGGACTA-3′, SEQ ID NO. 8, and an antisense primer 5′-ATGAGTAGCCAGGACTGAAGCTGT-3′, SEQ ID NO. 9, corresponding to nucleotides −4 to +19 and +535 to +512, respectively, relative to the UGT2B10 translation start site. The 539 bp PCR product was subjected to digestion with HinfI (New England Biolabs) at 37° C. for 3 h. An endogenous HinfI restriction enzyme site present within this PCR-amplified fragment acted as a control for enzyme digestion. A migration pattern of 426 and 113 bp is indicative of the polymorphic SNP^{199T} variant, whereas a migration pattern of 222, 204, and 113 bp is indicative of the wild-type SNP^{199G}.

Statistical Analysis.

Michaelis-Menten kinetic constants were determined using Prism Version 5 software (La Jolla, Calif.). The two-sample t-test (two-tailed) was used to compare kinetic values of glucuronide formation for the UGT1A4 and 2B10 isoforms against OLZ in cell lines and HLMs. To perform the most conservative comparison, unequal variances were assumed when comparing levels of OLZ-glucuronide formation in HLMs with wild-type alleles versus HLM with one or two polymorphic alleles. All the levels of OLZ-glucuronide formation were power transformed (using a power of 1/1.5) to make sure the data was approximately normally distributed. Analysis of OLZ glucuronidation in HLM stratified by UGT1A4 genotypes was performed only for those specimens also exhibiting the wild-type UGT2B10 (*1/*1) genotype (n=92); similarly, analysis of OLZ glucuronidation in HLM stratified by UGT2B10 genotypes was performed only for those specimens also exhibiting the wild-type UGT1A4 (*1/*1) genotype (n=95). Regression analysis of OLZ-glucuronide formation against genotype was performed (SAS Corporation, Cary, N.C.).

Characterization of OLZ Glucuronidation.

FIG. 1 outlines the enzymes previously shown to be involved in OLZ metabolism and the relative urinary contribution of each pathway (Devane, C. et al., Antipsychotics. 1st ed. Ambler: Lippencott Williams & Wilkins; 2000). In subjects taking OLZ, unchanged OLZ comprises ˜18% of total urinary OLZ; OLZ glucuronides comprise nearly 61% of all urinary OLZ metabolites, with the 10-N-glucuronide comprising ˜67% of urinary OLZ glucuronides (Kassahun, K. et al., Drug Metab Dispos. 1997 Jan. 1, 1997; 25(1):81-93; Calligaro, D. et al., Bioorganic and Medicinal Chemistry Letters. 1997; 7(0:25-30; Kassahun, K. et al., Drug Metab Dispos. 1998; 26(9):848-55).

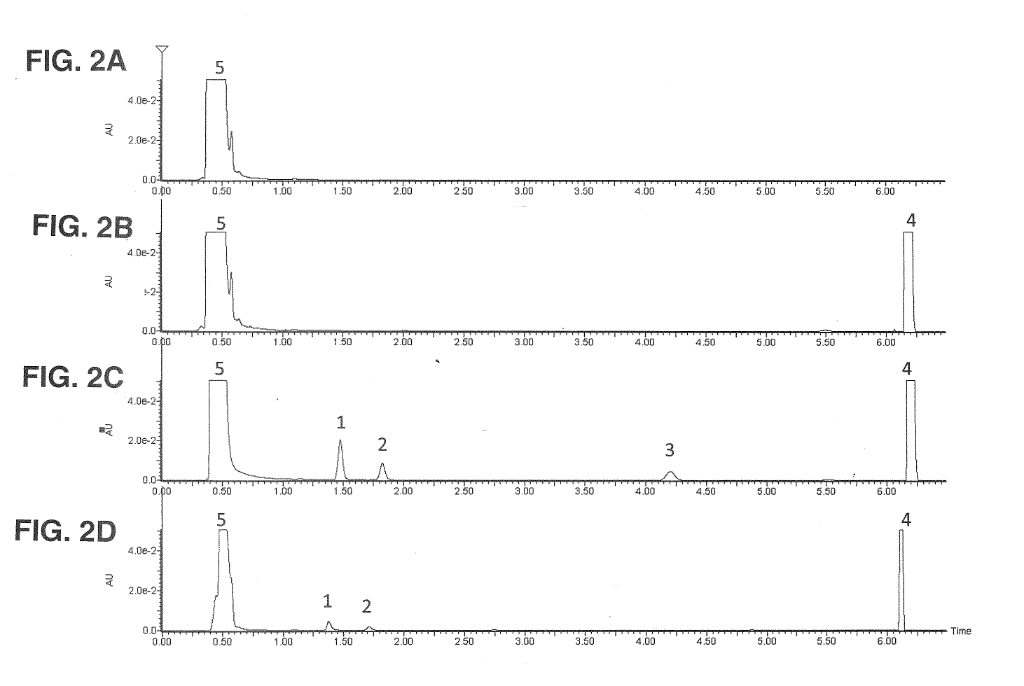

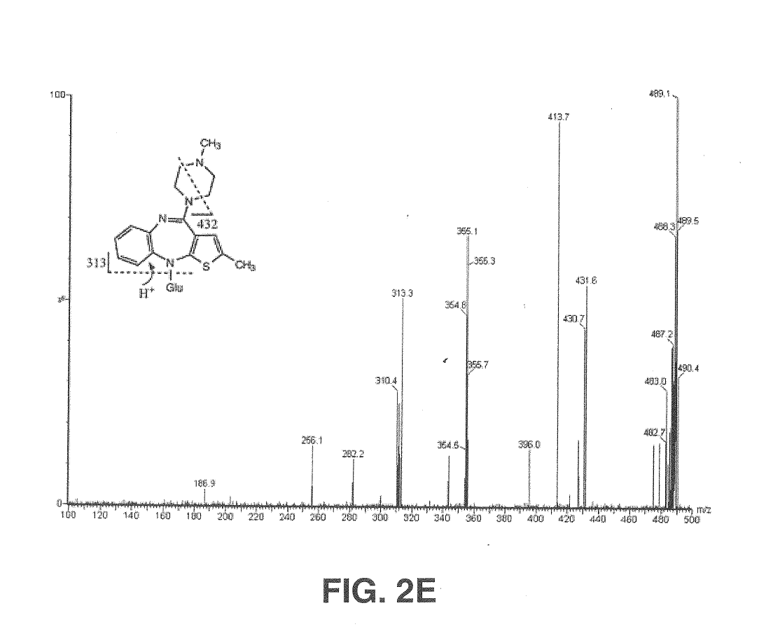

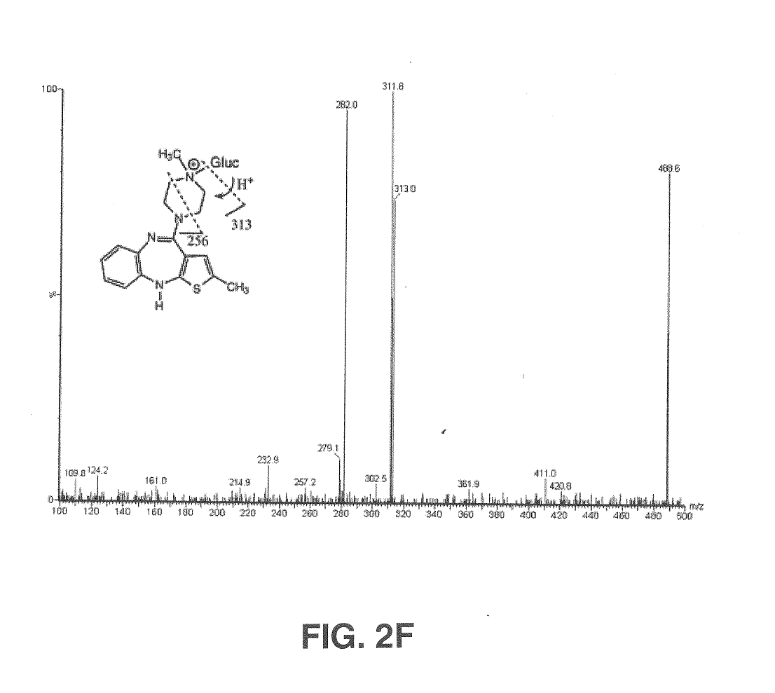

Glucuronidation assays were performed and OLZ glucuronides were separated by UPLC. In addition to an OLZ peak at a retention time of 6.2 min (FIG. 2B), incubations of OLZ with HLM yielded three peaks at 1.45 min, 1.78 min, and 3.83 min (FIG. 2C). While the 3.83 min peak was highly sensitive to treatment with β-glucuronidase (FIG. 2D), the peaks at 1.45 and 1.78 min were less sensitive to this treatment but were extremely sensitive to treatment with 3N HCl solution, a pattern that was previously reported for these OLZ glucuronides in Kassahun, K. et al., Drug Metab Dispos. 1997 Jan. 1, 1997; 25(1):81-93; and Kassahun, K. et al., Drug Metab Dispos. 1998; 26(9):848-55. The peaks at 1.45, 1.78, and 3.83 min all demonstrated a [M⁺] peak at m/z 489 (the glucuronide conjugate of OLZ) by mass spectrometry (MS/MS) analysis. The peaks at 1.45 and 1.78 min showed a [M+H]⁺ peak at m/z 313 for OLZ after loss of the glucuronic acid moiety (molecular weight=176 g/mol) and a m/z 432 fragment after loss of the CH₂═CH—NH—CH₃ methyl piperazine moiety (FIG. 2E). This is identical to the pattern observed for the two OLZ-10-N-glucuronide isomers reported previously in Kassahun, K. et al., Drug Metab Dispos. 1997 Jan. 1, 1997; 25(1):81-93; and Kassahun, K. et al., Drug Metab Dispos. 1998; 26(9):848-55. The peak at 183 min (FIG. 2F) demonstrated a [M+H]⁺ peak at m/z 313 (OLZ) and at m/z 282 due to the loss of CH₃NH₂ and glucuronic acid, but no peak at m/z 423, which is characteristic of the OLZ-4′-N-glucuronide as shown in Kassahun, K. et al., Drug Metab Dispos. 1997 Jan. 1, 1997; 25(1):81-93; and Kassahun, K. et al., Drug Metab Dispos. 1998; 26(9):848-55.

FIGS. 2A-2F show results of UPLC and MS/MS analysis of OLZ glucuronides formed by HLM. Glucuronidation assays were performed using 12.5 μg HLM protein and 300 μM OLZ, and incubated at 37° C. for 2 h with 4 mM UDP-glucuronic acid prior to analysis by UPLC as described in the Materials and Methods. Panel A, HLM+vehicle (1% DMSO); panel B, UGT-null HEK 293 cell homogenates+OLZ; panel C, HLM+OLZ; panel D, HLM+OLZ and 1,000 U of β-glucuronidase. Panel E, mass spectra of UPLC peaks 1 and 2; panel F, mass spectra of peak 3; Peak 1, OLZ-10-N-glucuronide isomer 1; peak 2, OLZ-10-N-glucuronide isomer 2; peak 3, OLZ-4′-N-glucuronide; peak 4, OLZ; peak 5, UDP-glucuronic acid. AU, absorbance.

Previous studies of selected UGT enzymes suggested that UGT1A4 was active against OLZ. To fully characterize all of the UGTs responsible for OLZ glucuronidation, a comprehensive screening of OLZ glucuronidation activity by homogenates from HEK293 cells over-expressing wild-type UGTs 1A1, 1A3, 1A4, 1A6, 1A7, 1A8, 1A9, 1A10, 2B4, 2B7, 2B10, 2B11, 2B15 and 2B17 was performed. Two UGTs exhibited detectable levels of activity against OLZ: the hepatic UGTs 1A4 and 2B10. None of the other UGTs screened in our assays (UGTs 1A1, 1A3, 1A6, 1A7, 1A8, 1A9, 1A10, 2B4, 2B7, 2B11, 2B15 or 2B17) exhibited any glucuronidation activity against OLZ using up to 250 μg of UGT-over-expressing cell homogenate. The UPLC 3-peak pattern and retention times for UGTs 1A4 and 2B10 were identical to that observed for HLM and showed similar sensitivity to treatment with β-glucuronidase and 3N HCl solution. Similar to that observed for OLZ glucuronides observed in incubations with HLM, the UGT1A4- and UGT2B10-generated peaks were identified as OLZ-10-N-glucuronide isomer 1 at 1.45 min, OLZ-10-N-glucuronide isomer 2 at 1.78 min, and OLZ-4′-N-glucuronide at 3.83 min as determined by UPLC/MS/MS analysis.

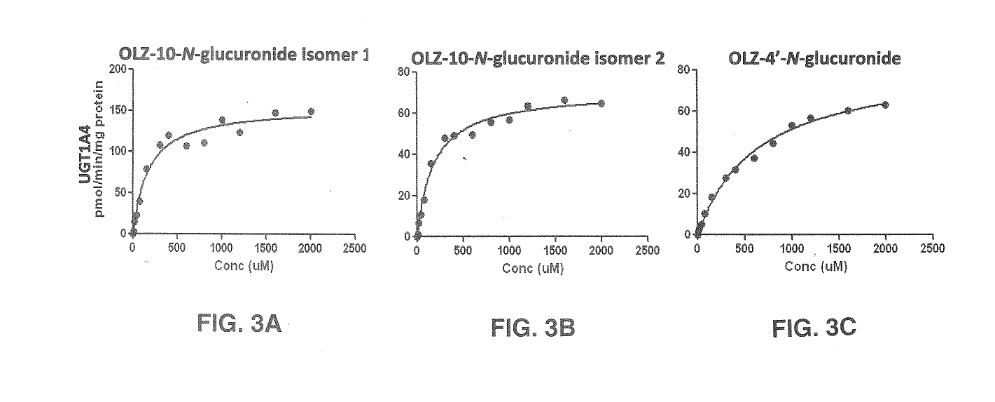

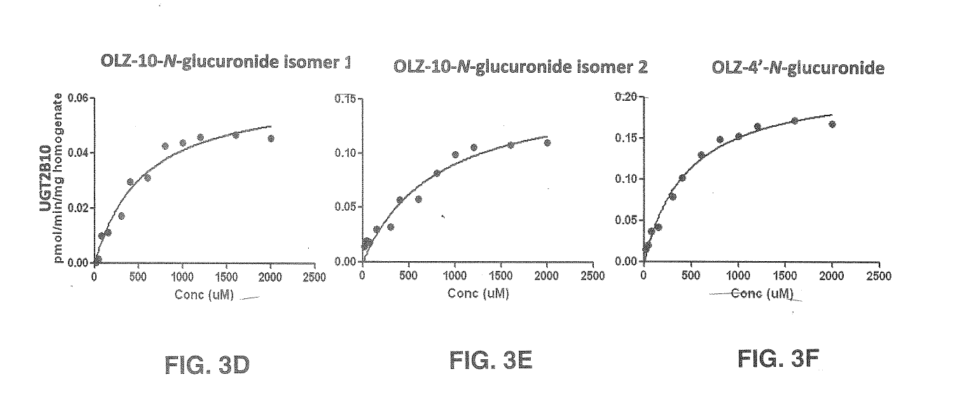

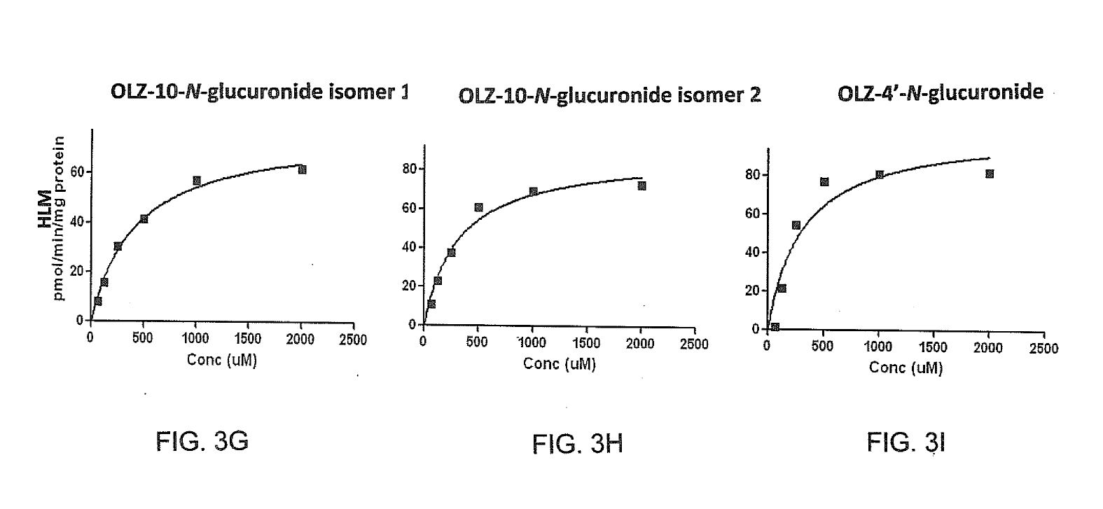

Representative kinetic plots of glucuronidation rate versus substrate concentration are shown in FIGS. 3A-1 for wild-type UGTs 1A4 (UGT1A4^{24Pro48Leu}) and 2B10 (UGT2B10^{67Asp}) as well as HLMs with wild-type UGTs 1A4 and 2B10. FIGS. 3A-3I are kinetic curves for wild-type UGT1A424Pro/48Leu- and UGT2B1067Asp-over-expressing cell homogenates and wild-type HLM against OLZ. Representative kinetic curves were obtained by performance of glucuronidation assays as described herein using OLZ concentrations of 9.4, 18.8, 37.6, 75, 150, 300, 400, 600, 800, 1000, 1200, 1600, and 2000 μM for cell lines and 62.5, 125, 250, 500, 1000, and 2000 μM for HLM. Wild-type HLM were from subjects exhibiting the UGT1A4(*1/*1)/UGT2B10(*1/*1) genotype. UGT2B10 exhibited a significantly (p<0.0001) decreased binding affinity against OLZ (K_{M}=564±15 μM) as compared to UGT1A4 (K_{M}=156±17 μM) for the formation of OLZ-10-N-glucuronide isomer 1 and for OLZ-10-N-glucuronide isomer 2, K_{M}=818±23 μM vs. 206±37 μM, respectively; p<0.0001; as shown in Table I.

TABLE I
Kinetic analysis of UGT1A4 and UGT2B10
variants against OLZ in vitro.

| | | | |
|---|---|---|---|
| | Vₘₐₓ | | Vₘₐₓ/K_{M}^{a} |
| | (pmol · min⁻¹ · | K_{M} | (nl · min⁻¹ · |
| UGT variant | mg⁻¹) | (μM) | mg⁻¹) |

OLZ-10-N-glucuronide isomer 1

| | | | |
|---|---|---|---|
| UGT1A4^{24Pro/48Leu} | 145 ± 12 | 156 ± 17 | 0.94 ± 0.03 |
| UGT1A4^{24Thr/48Lcu} | 180 ± 24 | 584 ± 67 | 0.31 ± 0.07*^{†} |
| UGT1A4^{24Pro/48Val} | 223 ± 5 | 69 ± 6.5 | 3.4 ± 0.3* |
| UGT2B10^{67Asp} | 0.35 ± 0.19 | 564 ± 15 | 0.0006 ± 0.001 |

| | |
|---|---|
| UGT2B10^{67Tyr} | no activity detected |

OLZ-10-N-glucuronide isomer 2

| | | | |
|---|---|---|---|
| UGT1A4^{24Pro/48Leu} | 73 ± 5 | 206 ± 37 | 0.36 ± 0.06 |
| UGT1A4^{24Thr/48Leu} | 61 ± 16 | 152 ± 23 | 0.40 ± 0.04^{†} |
| UGT1A4^{24Pro/48Val} | 187 ± 18 | 110 ± 14 | 1.7 ± 0.1 |
| UGT2B10^{67Asp} | 0.93 ± 0.03 | 818 ± 23 | 0.001 ± 0.0001 |

| | |
|---|---|
| UGT2B10^{67Tyr} | no activity detected |

OLZ-4′-N-glucuronide

| | | | |
|---|---|---|---|
| UGT1A4^{24Pro/48Leu} | 105 ± 23 | 810 ± 135 | 0.13 ± 0.01 |
| UGT1A4^{24Thr/48Leu} | 60 ± 16 | 358 ± 54 | 0.17 ± 0.05 |
| UGT1A4^{24Pro/48Val} | 47 ± 5 | 326 ± 33 | 0.15 ± 0.02 |
| UGT2B10^{67Asp} | 1.2 ± 0.1 | 457 ± 11** | 0.003 ± 0.0002 |

| | |
|---|---|
| UGTZB10^{67Tyr} | no activity detected |

^{a}For UGT2B10 variants, value represents K_{M}/Vₘₐₓ due to lack of availability of an UGT2B10 antibody to accurately quantify UGT2B10 protein levels.
*p < 0.0001,
**p = 0.0018 versus corresponding value for UGT1A4^{24Pro/48Leu};
^{†}p < 0.0001 versus corresponding value for UGT1A4^{24Pro/48Val}.

A significantly (p=0.0018) higher binding affinity was observed for UGT2B10 (K_{M}=457±11 μM) as compared to UGT1A4 (K_{M}=810±135 μM) when forming the OLZ-4′-N-glucuronide.

The K_{M} exhibited by HLM with wild-type UGTs 1A4 and 2B10 for both OLZ-10-N-glucuronide isomer 1 and for OLZ-10-N-glucuronide isomer 2 formation as shown in Table II, were between the K_{M}'s exhibited by homogenates from UGT1A4 and UGT2B10-over-expressing cells. The K_{M} exhibited by these HLM for OLZ-4′-N-glucuronide formation was similar to that observed for homogenates from UGT2B10-over-expressing cells.

TABLE II
Kinetic analysis of OLZ glucuronidation in HLMs
of varying UGT1A4 and UGT2B10 genotypes

| | | | |
|---|---|---|---|
| | Vₘₐₓ | K_{M} | Vₘₐₓ/K_{M} |
| HLM genotype | (pmol · min⁻¹ · mg⁻¹) | (μM) | (nl · min⁻¹ · mg⁻¹) |

OLZ-10-N-glucuronide isomer 1

| | | | |
|---|---|---|---|
| UGT1A4(*1/*1)/ | 94.0 ± 14 | 352 ± 59 | 0.28 ± 0.08 |
| UGT2B10(*1/*1)^{a} | | | |
| UGT1A4(*1/*3)/ | 160 ± 14* | 328 ± 33 | 0.50 ± 0.08** |
| UGT2B10(*1/*1)^{a} | | | |
| UGT1A4(*3/*3)/ | 188 ± 47^{†} | 268 ± 41 | 0.70 ± 0.07^{††} |
| UGT2B10(*1/*1)^{a} | | | |
| UGT2B10(*1/*2)/ | 56 ± 23\hat{ }\hat{ } | 302 ± 50 | 0.19 ± 0.08\hat{ }\hat{ }\hat{ } |
| UGT1A4(*1/*1)^{b} | | | |
| UGT2B10(*2/*2)/ | 69 | 273 | 0.25 |
| UGT1A4(*1/*1)^{b} | | | |

OLZ-10-N-glucuronide isomer 2

| | | | |
|---|---|---|---|
| UGT1A4(*1/*1)/ | 63 ± 23 | 311 ± 27 | 0.2 ± 0.07 |
| UGT2B10(*1/*1)^{a} | | | |
| UGTlA4(*1/*3)/ | 73 ± 17 | 239 ± 22*** | 0.31 ± 0.08 |
| UGT2B10(*1/*1)^{a} | | | |
| UGT1A4(*3/*3)/ | 106 ± 16 | 263 ± 86 | 0.42 ± 0.08 |
| UGT2B10(*1/*1)^{a} | | | |
| UGT2B10(*1/*2)/ | 30 ± 14⁺ | 196 ± 54** | 0.16 ± 0.09 |
| UGT1A4(*1/*1)^{b} | | | |
| UGT2B10(*2/*2)/ | 41.5 | 271 | 0.15 |
| UGT1A4(*1/*1)^{b} | | | |

OLZ-4′-N-glucuronide

| | | | |
|---|---|---|---|
| UGT1A4(*1/*1)/ | 82 ± 20 | 317 ± 39 | 0.26 ± 0.07 |
| UGT2B10(*1/*1)^{a} | | | |
| UGT1A4(*1/*3)/ | 70 ± 24**** | 263 ± 59 | 0.26 ± 0.04***** |
| UGT2B10(*1/*1)^{a} | | | |
| UGT1A4(*3/*3)/ | 156 ± 18^{†††} | 337 ± 37 | 0.46 ± 0.01\hat{ } |
| UGT2B10(*1/*1)^{a} | | | |
| UGT2B10(*1/*2)/ | 47 ± 20⁺ | 602 ± 57^{+,++} | 0.08 ± 0.03***^{,+++} |
| UGT1A4(*1/*1)^{b} | | | |
| UGT2B10(*2/*2)/ | 37 | 796 | 0.047 |
| UGT1A4(*1/*1)^{b} | | | |

^{a}Three HLM with the UGT1A4 (*1/*1) genotype, three HLM with the UGT1A4 (*1/*3) genotype, and two HLM with the UGT1A4 (*3/*3) genotype were examined in this analysis. Only HLM with the UGT2B10 (*1/*1) genotype was used to compare varying UGT1A4 genotypes. HLMs of each genotype were chosen at random.
^{b}Three HLM with the UGT2B10 (*1/*2) genotype and the single HLM with the UGT2B10 (*2/*2) genotype were examined in this analysis. Only HLM with the UGT1A4 (*1/*1) genotype were used to compare varying UGT2B10 genotypes.
*p = 0.0046,
**p = 0.03,
***p = 0.02,
^{†}p = 0.04,
^{††}p = 0.009,
^{†††}p = 0.028,
\hat{ }p = 0.0078, and
⁺⁺p = 0.002 versus the corresponding value for HLM with the UGT1A4 (*1/*1)/UGT2B10 (*1/*1) genotype.
****p = 0.03,
*****p = 0.005,
\hat{ }\hat{ }p = 0.02,
\hat{ }\hat{ }\hat{ }p = 0.005,
⁺p = 0.01, and
⁺⁺⁺p = 0.0004 versus the corresponding value for HLM with the UGT1A4 (*3/*3)/UGT2B10 (*1/*1) genotype.

For UGT1A4, UGT1A4 *1/*1 in Table II indicates that UGT1A4 is either proline or threonine at amino acid 24 and is leucine at amino acid 48.

Variants of UGT1A4 at amino acid 48 are indicated in Table II as: UGT1A4 *1/*3 indicating the genotype of the individual is UGT1A4^{48L} on one allele and UGT1A4^{48V} on one allele or UGT1A4 *3/*3 indicating the genotype of the individual is UGT1A4^{48V} on both alleles.

Wild-type for UGT2B10 at amino acid 67 is indicated in Table II as UGT2B10 *1/*1 indicating the genotype of the individual is UGT2B10^{67D} on both alleles.

Variants of UGT2B10 at amino acid 67 are indicated in Table II as UGT2B10 *1/*2 indicating the genotype of the individual is UGT2B10^{67Y} on one allele or UGT2B10 *2/*2 indicating the genotype of the individual is UGT2B10^{67Y} on both alleles.

Analysis of OLZ Glucuronidation by Hepatic UGT Variants

UGT1A4 is a hepatic enzyme that exhibits prevalent functional missense polymorphisms as described in Sun, D. et al., Breast Cancer Res. 2006; 8(4):R50; Erichsen, T. et al., Toxicology and Applied Pharmacology. 2008; 230(2):252-60; and Wiener, D., Cancer Res. 2004 Feb. 1; 64(3):1190-6. HEK293 cell lines over-expressing the UGT1A4^{24Thr/48Leu} and UGT1A4^{24Pro/48Val} variants are described in Blevins-Primeau, A. et al., Cancer Res. 2009 Mar. 1, 2009; 69(5):1892-900; Wiener, D. et al., Drug Metab Dispos. 2004 January; 32(1):72-9; Chen, G. et al., Pharmacogenetics and Genomics 2008; 18(3):181-91; Dellinger, R. et al., Drug Metabolism and Disposition. 2006 June 2006; 34(6):943-9; Chen, G. et al., Cancer Research. 2007 Oct. 1, 2007; 67(19):9024-9; and Wiener, D., Cancer Res. 2004 Feb. 1; 64(3):1190-6. After normalization of UGT1A4 variant protein levels in UGT1A4-over-expressing cell homogenates by Western blot analysis, no significant difference in Vₘₐₓ/K_{M} for OLZ-4′-N-glucuronide formation was observed between the UGT1A4^{24Pro/48Val} variant and either of the other two UGT1A4 isoforms (Table I). The Vₘₐₓ/K_{M} of the UGT1A4^{24Pro/48Val} variant was 3.6- (p<0.0001) and 11- (p<0.0001) fold higher than that observed for wild-type UGT1A4^{24Pro/48Leu} and the UGT1A4^{24Thr/48Leu} variant, respectively, for formation of OLZ-10-N-glucuronide isomer 1, and 4.7- (p<0.0001) and 4.3- (p<0.0001) fold higher, respectively, for OLZ-10-N-glucuronide isomer 2 formation (Table I). While a significant (p<0.0001) 3-fold lower Vₘₐₓ/K_{M} was observed for the UGT1A4^{24Thr/48Leu} variant versus the wild-type UGT1A4^{24Pro/48Leu} for formation of OLZ-10-N-glucuronide isomer 1, no difference was observed for OLZ-10-N-glucuronide isomer 2 or the OLZ-4′-N-glucuronide.

Similar to that described for UGT1A4, a prevalent missense polymorphism also exists for UGT2B10 at codon 67 A HEK293 cell line over-expressing the UGT2B10^{67Tyr} variant has been described in Chen, G. et al., Pharmacogenetics and Genomics 2008; 18(3):181-91; and Chen, G. et al., Cancer Research. 2007 Oct. 1, 2007; 67(1.9):9024-9. Real-time PCR demonstrated similar levels of UGT2B10 expression in the UGT2B10 wild-type and codon 67 variant-over-expressing cell lines, which were in agreement with levels previously published for these cell lines, described in Chen, G. et al., Pharmacogenetics and Genomics 2008; 18(3):181-91; and Chen, G. et al., Drug Metab Dispos. 2008 May 1, 2008; 36(5):824-30. Unlike the activity observed for the wild-type UGT2B10^{67Asp}, no glucuronidation activity was observed for the UGT2B10^{67Tyr} variant against OLZ (Table I).

Analysis of OLZ Glucuronidation by HLM Stratified by UGT1A4 or UGT2B10 Genotypes.

The prevalence of the UGT1A4^{24Thr/48Leu}, UGT1A4^{24Pro/48Val} and UGT2B10^{67Tyr} variants is ˜8%, 9%, and 10% in Caucasians, respectively as described in Chen, G. et al., Pharmacogenetics and Genomics 2008; 18(3):181-91; Chen, G. et al., Cancer Research. 2007 Oct. 1, 2007; 67(19):9024-9; Erichsen, T. et al., Toxicology and Applied Pharmacology. 2008; 230(2):252-60; and Wiener, D., Cancer Res. 2004 Feb. 1; 64(3):1190-6. A series of 105 HLM were examined for their glucuronidation activity against OLZ to determine the in vivo relationship between OLZ glucuronidation and the UGT1A4 and UGT2B10 polymorphisms. The rate of OLZ-glucuronide formation was determined by UPLC using 300 μM OLZ, a concentration within the linear range of kinetic analysis for HLM against OLZ. Comparing HLM homozygous for the wild-type UGT2B10^{67Asp} allele [UGT2B10 (*1/*1) genotype (n=92)], no significant difference in formation of any OLZ glucuronide was observed when comparing those homozygous for the UGT1A4^{24Thr/48Leu} allele [UGT1A4 (*2/*2) genotype; n=2] to HLM homozygous for the UGT1A4^{24Pro/48Leu} allele [UGT1A4 (*1/*1) genotype; n=73], even after excluding HLM with one or more UGT1A4*3 (UGT1A4^{24Pro/48Val}) alleles. Therefore, for further analysis of the UGT1A4*3 and UGT2B10^{67Tyr} (UGT2BIO*2) alleles, ELM were not sub-stratified based on the UGT1A4*2 allele.

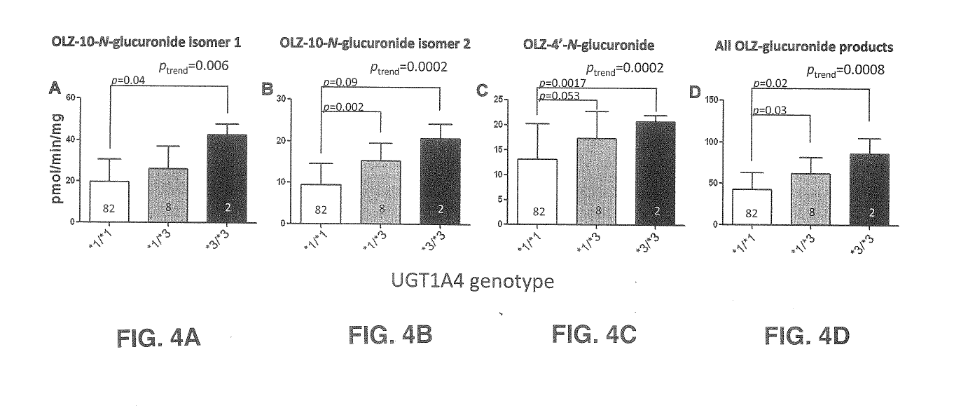

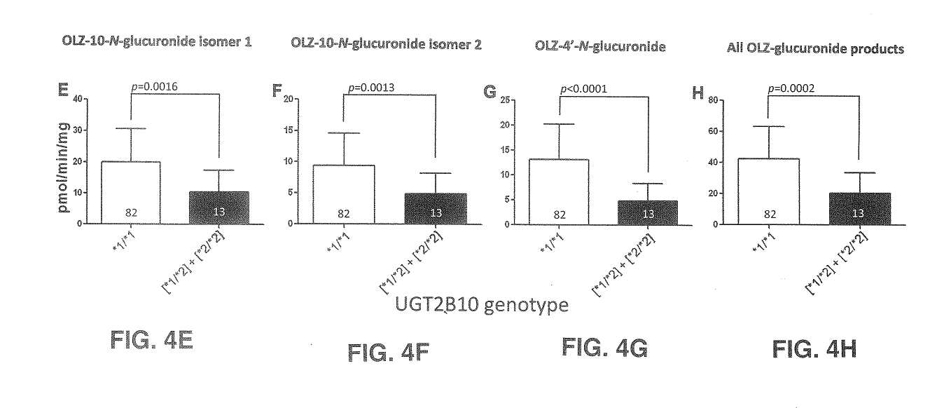

FIGS. 4A-4H show HLM activity stratified by UGT genotypes. Shown are the levels of OLZ-10-N-glucuronide isomer 1, OLZ-10-N-glucuronide isomer 2, and OLZ-4′-N-glucuronide formation versus UGT1A4 or UGT2B10 genotypes in HLM. Glucuronidation activity assays were performed using 300 μM OLZ and 12.5 μg of HLM protein, and OLZ glucuronides were detected and separated by UPLC as described in the Materials and Methods. Using genomic DNA from the same liver specimens for which HLMs were prepared, UGT1A4 and UGT2B10 genotypes were determined using DNA sequencing and RFLP analysis, respectively. Panels A-D, OLZ glucuronidation in ELM stratified by UGT1A4 genotypes; panels E-H, OLZ glucuronidation in HLM stratified by UGT2B10 genotypes. Panel A and E, OLZ-10-N-glucuronide isomer 1 formation; panels B and F, OLZ-10-N-glucuronide isomer 2 formation; panels C and G, OLZ-4′-N-glucuronide formation; panels D and H, all OLZ-glucuronide product formation. Analysis of OLZ glucuronidation in ELM stratified by UGT1A4 genotypes was performed only for those specimens also exhibiting the wild-type UGT2B10 (*1/*1) genotype (n=92); similarly, analysis of OLZ glucuronidation in ELM stratified by UGT2B10 genotypes was performed only for those specimens also exhibiting the wild-type UGT1A4 (*WI) genotype (n=95).

For HLM homozygous for the wild-type UGT2B10^{67Asp} (*1/*1) genotype, there was a significant 2.1- (p=0.04) and 1.6- (p=0.0017) fold increase in formation of the OLZ-10-N-glucuronide isomer 1 and the OLZ-4′-N-glucuronide, respectively (FIGS. 4A and 4C) and a significant (p=0.02) 2.0-fold increase in overall OLZ glucuronidation activity (FIG. 4D) in HLM with the UGT1A4 (*31*3) genotype (n=2) as compared with HLM with the UGT1A4 (*1/*1) genotype (n=82). While a 2.2-fold increase in OLZ-10-N-glucuronide isomer 2 formation was observed for ELM with the UGT1A4 (*3/*3) genotype versus HLM with the UGT1A4 (*1/*1) genotype, this difference was not significant (p=0.09). Regression analysis showed that the UGT1A4 *3 allele is a significant predictor for formation of the OLZ-10-N-glucuronide isomer 1 (p=0.0047), the OLZ-10-N-glucuronide isomer 2 (p=0.0001), the OLZ-4′-N-glucuronide (p=0.02), and overall OLZ glucuronidation (p=0.0013). For each UGT1A4 *3 allele, product formation increased by 3.0, 3.3, 1.2, and 7.9 pmol·min⁻¹·mg protein⁻¹ for OLZ-10-N-glucuronide isomer 1, OLZ-10-N-glucuronide isomer 2, OLZ-4′-N-glucuronide, and overall glucuronidation, respectively, representing a 16, 25, 10, and 15% increase in glucuronidation for each UGT1A4 *3 allele compared to the mean glucuronide-product formation by wild-type samples, respectively.

For HLM with the wild-type UGT1A4 (*1/*1) genotype (n=95), there was only a single HLM with the UGT2B10 (*2/*2) genotype. Therefore, HLM with the UGT2B210 (*1/*2) (n=12) or (*2/*2) (n=1) genotypes were combined into one group for analysis of OLZ glucuronidation activity stratified by UGT2B10 genotype. There was a significant 1.9-fold decrease in both OLZ-10-N-glucuronide isomer 1 (p=0.0016; FIG. 4E) and isomer 2 (p=0.0013; FIG. 4F) formation, a significant 2.7-fold decrease in OLZ-4′-N-glucuronide formation (p<0.0001; FIG. 4G), and a significant 2.1-fold decrease in overall OLZ glucuronidation (p=0.0002; panel H) in UGT1A4 (*1/*1) HLMs with at least one UGT2B10*2 allele as compared to UGT1A4 (*1/*1) HLMs with the UGT2B10 (*1/*1) genotype. The single HLM with a combined UGT1A4 (*1/*1)/UGT2B10 (*2/*2) genotype exhibited a 2.6-, 2.1-, and 4.1-fold decrease in the formation of OLZ-10-N-glucuronide isomer 1, OLZ-10-N-glucuronide isomer 2, and OLZ-4′-N-glucuronide, respectively, as compared to UGT1A4 (*1/*1) HLMs with the UGT2B10 (*1/*1) genotype, suggesting a trend towards decreasing OLZ glucuronidation activity with increasing numbers of the UGT2B10*2 allele. Regression analysis showed that the UGT2B10*2 allele was a significant predictor for formation of the OLZ-10-N-glucuronide isomer 1 (p=0.002), the OLZ-10-N-glucuronide isomer 2 (p=0.0019), the OLZ-4′-N-glucuronide (p<0.0001), and overall OLZ glucuronidation (p<0.0001). For each UGT2B10*2 allele, product formation decreased by 4.1, 2.0, 4.3, and 10.5 pmol·min⁻¹·mg protein⁻¹ for OLZ-10-N-glucuronide isomer 1, OLZ-10-N-glucuronide isomer 2, OLZ-4′-N-glucuronide, and overall glucuronidation, respectively, representing a 20, 20, 50, and 30% decrease in glucuronide formation for each UGT2B10*2 allele compared to the mean glucuronide-product formed by wild-type samples, respectively. There was insufficient power to examine the effect of combined UGT1A4 and UGT2B10 genotypes on OLZ glucuronidation in this series of HLM specimens.

To better assess the relative contribution of UGT1A4 to OLZ glucuronidation, HLM were co-incubated with 300 μM OLZ and 2000 μM of the UGT1A4-specific inhibitor, LTG (Linnet, K. et al., Hum Psychopharmacol. 2002 July; 17(5):233-8; Rowland, A. et al., Drug Metabolism and Disposition. 2006 Jun. 1, 2006; 34(6):1055-62. Overall OLZ glucuronidation was inhibited by 12±3.4% in UGT1A4 (*1/*1)/UGT2B10 (*1/*1) HLM incubated with LTG. Using assay conditions (25 μM OLZ and 2000 μM LTG) similar to those described in Linnet, K. et al., Hum Psychopharmacol. 2002 July; 17(5):233-8, overall OLZ glucuronidation was inhibited by 41% in homogenates from UGT1A4-over-expressing cells.

Results of kinetic analysis of HLM with varying UGT1A4 and UGT2B10 genotypes are shown in Table H. For HLM stratified by UGT1A4 genotypes, there was a significant 1.8- (p=0.03) and 2.5- (p=0.009) fold increased Vₘₐₓ/K_{M} for formation of the OLZ-10-N-glucuronide isomer 1 in UGT2B10 (*1/*1) HLM with the UGT1A4 (*1/*3) and UGT1A4 (*31*3) genotypes, respectively, as compared to UGT2B10 (*1/*1) HLM with the UGT1A4 (*1/*1) genotype. This was manifested primarily via an increase in Vₘₐₓ, with HLM with the UGT1A4 (*1/*3)/UGT2B10 (*1/*1) and UGT1A4 (3/*3)/UGT2B10 (*1/*1) genotypes exhibiting a significant 1.7- (p=0.0046) and 2.0- (p=0.04) fold higher Vₘₐₓ, respectively, for OLZ-10-N-glucuronide isomer 1. There was a significant trend of increasing activity as measured by Vₘₐₓ/K_{M} with increasing numbers of the UGT1A4*3 allele for formation of the OLZ-10-N-glucuronide isomer 1 (p<0.0001). For formation of the OLZ-10-N-glucuronide isomer 2, there was a significant (p=0.008) trend of increasing Vₘₐₓ/K_{M} with increasing numbers of the UGT1A4*3 allele (Table II). For the OLZ-4′-N-glucuronide, HLM with the UGT1A4 (*3/*3)/UGT2B10 (*1/*1) genotype exhibited a 1.8-fold higher Vₘₐₓ/K_{M} (p=0.0078), manifested primarily via a 1.9-fold higher Vₘₐₓ (p=0.028), as compared to HLM with the UGT1A4 (*1/*1)/UGT2B10 (*1/*1) genotype (Table II). A significant (p=0.0075) trend of increasing Vₘₐₓ/K_{M} for HLM with increasing numbers of the UGT1A4*3 allele was also observed.

The effect of UGT2B10 genotype on HLM glucuronidation activities was strongest for the OLZ-4′-N-glucuronide. There was a significant 3.3-fold lower Vₘₐₓ/K_{M} (p=0.02) for formation of the OLZ-4′-N-glucuronide in HLM with the UGT1A4 (*1/*1)/UGT2B10 (*1/*2) genotype as compared to HLM with the UGT1A4 (*1/*1)/UGT2B10 (*1/*1) genotype, an effect that was manifested by primarily by a significant decrease in Vₘₐₓ (p=0.01; Table II). A significant (p=0.0004) 5.8-fold lower Vₘₐₓ/K_{M} was observed for HLM with the UGT1A4 (*1/*1)/UGT2B10 (*1/*2) genotype when compared to HLM with the UGT1A4 (*3/*3)/UGT2B10 (*1/*1) genotype. While statistical analysis could not be performed for comparisons with the single HLM with the UGT1A4 (*1/*1)/UGT2B10 (*2/*2) genotype, this HLM exhibited a 1.7-, 5.5- and 9.8-fold lower Vₘₐₓ/K_{M} for formation of the OLZ-4′-N-glucuronide as compared to HLM with the UGT1A4 (*1/*1)/UGT2B10 (*11*2), UGT1A4 (1/*1)/UGT2B10 (*1/*1) and UGT1A4 (*3/*3)/UGT2B10 (*1/*1) genotypes, respectively.

While significant alterations in OLZ-10-N-glucuronide isomer 1 kinetics were not observed when comparing UGT1A4 (*1/*1)/UGT2B10 (*11*2) versus UGT1A4 (*1/*1)/UGT2B10 (*1/*1) HLM, a significant (p=0.005) 3.7-fold decrease in Vₘₐₓ/K_{M} was observed when comparing UGT1A4 (*1/*1)/UGT2B10 (*1/*2) versus UGT1A4 (*3/*3)/UGT2B10 (*1/*1) HLM. A similar pattern was observed for OLZ-10-N-glucuronide isomer 2, with HLM with the UGT1A4 (*1/*1)/UGT2B10 (*11*2) genotype exhibiting a near-significant (p=0.057) 2.6-fold lower Vₘₐₓ/K_{M} as compared to HLM with the UGT1A4 (*3/*3)/UGT2B10 (*1/*1) genotype. In both cases these differences were primarily due to significant (p<0.02) decreases in Vₘₐₓ.

UGT1A4 amino acid 24 variant (P24T, identified herein as UGT1A4*2 allele): This variant decreases the affinity and efficiency of the UGT1A4 enzyme in forming the OLZ-10-N-glucuronide isomer 1 compared to wild-type UGT1A4 and decreases the affinity and efficiency of the UGT1A4 enzyme in forming the OLZ-10-N-glucuronide isomers 1 and 2 compared to the amino acid 48 variant UGT1A4^{24P48V}.

UGT1A4 amino acid 48 variant (L48V, identified herein as UGT1A4 *3 allele): This variant increases the affinity and efficiency of the UGT1A4 enzyme. In human liver microsomes, this variant increases the rate and product formation of OLZ-10-N-glucuronide isomer 1 and OLZ-4′-N-glucuronide formation and overall glucuronide formation.

UGT2B10 amino acid 67 variant (D67Y, identified herein as UGT2B10 *2 allele): This variant is a functional knock-out of UGT2B10 glucuronidation activity towards olanzapine. In human liver microsomes, this variant decreases the rate of formation of both isomers of the OLZ-10-N-glucuronide, the OLZ-4′-N-glucuronide and overall OLZ-glucuronide formation.

Combined effect of the UGT1A4 amino acid 48 and UGT2B10 amino acid 67 variants in human liver microsomes is likely to alter glucuronidation by decreased glucuronidation of olanzapine compared to wild-type (UGT1A4 (*1/*1)/UGT2B10 (*1/*1) in individuals with the UGT1A4 (*3/*3)/UGT2B10 (*2/*2) genotype

UGT2B10 is a well-known UDP glucuronosyltransferase, see for example, Jin C J, et al., Biochemical and Biophysical Research Communications. 1993; 194(1):496-503; Kerdpin O, et al., Drug Metabolism and Disposition. 2009 Sep. 1, 2009; 37(9):1948-55; and Beaulieu M, et al., Biochemical and Biophysical Research Communications. 1998; 248(1):44-50.

A human UGT2B10 amino acid sequence is shown herein as SEQ ID No. 1 and a nucleic acid sequence encoding the UGT2B10 protein of SEQ ID No. 1 is shown herein as SEQ ID No. 2. In humans the UGT2B10 gene is on chromosome: 4; location: 4q13.2.

UGT2B10 Amino acid sequence SEQ ID NO. 1 (amino acid 67 D underlined and in bold):

| | |
|---|---|
| 1 | malkwttvll iqlsfyfssg scgkvlvwaa eyslwmnmkt ilkelvqrgh evtvlassas |
| 61 | ilfdpndsst lklevyptsl tktefeniim qlvkrlseiq kdtfwlpfsq eqeilwaind |
| 121 | iirnfckdvv snkklmkklq esrfdivfad aylpcgella elfnipfvys hsfspgysfe |
| 181 | rhsggfifpp syvpvvmskl sdqmtfmerv knmlyvlyfd fwfqifnmkk wdqfysevlg |
| 241 | rpttlsetmr kadiwlmrns wnfkfphpfl pnvdfvgglh ckpakplpke meefvqssge |
| 301 | ngvvvfslgs mvsnmteera nviatalaki pqkvlwrfdg nkpdalglnt rlykwipqnd |
| 361 | llghpktraf ithggangiy eaiyhgipmv giplffdqpd niahmkakga avrvdfntms |
| 421 | stdllnalkt vindpsyken imklsriqhd qpvkpldrav fwiefvmrhk gakhlrvaah |
| 481 | nltwfqyhsl dvigfllacv atvlfiitkc clfcfwkfar kgkkgkrd |

UGT2B10 Nucleic acid sequence SEQ ID NO. 2 (START codon and codon 67 underlined and in bold):

| | |
|---|---|
| 1 | aaaagaatta tcacattgca caaggatggc tctgaaatgg actacagttc tgctgataca |
| 61 | actcagtttt tactttagct ctgggagttg tggaaaggtg ctggtatggg ccgcagaata |
| 121 | cagcctttgg atgaatatga agacaatcct gaaagaactt gttcagagag gtcatgaggt |
| 181 | gactgtactg gcatcttcag cttccattct ttttgatccc aacgactcat ccactcttaa |
| 241 | acttgaagtt tatcctacat ctttaactaa aactgaattt gagaatatca tcatgcaatt |
| 301 | ggttaagaga ttgtcagaaa ttcaaaaaga tacattttgg ttaccttttt cacaagaaca |
| 361 | agaaatcctg tgggcaatta atgacataat tagaaacttc tgtaaagatg tagtttcaaa |
| 421 | taagaaactt atgaaaaaac tacaagagtc aagatttgac atcgtttttg cagatgctta |
| 481 | tttaccctgt ggtgagctgc tggctgagct atttaacata ccctttgtgt acagtcacag |
| 541 | cttcagtcct ggctactcat ttgaaaggca cagtggagga tttattttcc ctccttccta |
| 601 | cgtacctgtt gttatgtcaa aattaagtga tcaaatgact ttcatggaga gggtaaaaaa |
| 661 | tatgctctat gtgctttatt ttgacttttg gttccaaata tttaatatga agaagtggga |
| 721 | tcagttttac agtgaagttt taggaagacc cactacatta tctgagacaa tgaggaaagc |
| 781 | tgacatatgg cttatgcgaa actcctggaa ttttaaattt cctcatccat tcttaccaaa |
| 841 | tgttgatttt gttggaggac tccactgcaa acctgccaaa cccctaccta aggaaatgga |
| 901 | ggagtttgta cagagctctg gagaaaatgg tgttgtggtg ttttctctgg ggtcaatggt |
| 961 | cagtaacatg acagaagaaa gggccaacgt aattgcaaca gcccttgcca agatcccaca |
| 1021 | aaaggttctt tggagatttg atgggaataa accagatgcc ttaggtctca atactcgact |
| 1081 | gtacaagtgg ataccccaga atgaccttct aggtcatcca aaaaccagag cttttataac |
| 1141 | tcatggtgga gccaatggca tctatgaggc aatctaccat gggatcccta tggtgggcat |
| 1201 | tccattgttt tttgatcaac ctgataatat tgctcacatg aaggccaagg gagcagctgt |
| 1261 | tagagtggac ttcaacacaa tgtcgagtac agacctgctg aatgcactga agacagtaat |
| 1321 | taatgatcct tcatataaag agaatattat gaaattatca agaattcaac atgatcaacc |
| 1381 | agtgaagccc ctggatcgag cagtcttctg gattgaattt gtcatgcgcc acaaaggagc |
| 1441 | caaacatctt cgagttgcag cccacaacct cacctggttc cagtaccact ctttggatgt |
| 1501 | gattgggttc ctgctggctt gtgtggcaac cgtgctattt atcatcacaa agtgttgtct |
| 1561 | gttttgtttc tggaagtttg ctagaaaagg aaagaaggga aaaagggatt agttatatct |
| 1621 | gagatttgaa gctggaaaac ctgatagata ggaatacttc agttgattcc agcaataaat |
| 1681 | attgtgatgc aagatttctt tcttcctgtg acaaaaaaaa atcctttcga agtctacctt |
| 1741 | gtcaagtaaa aatttgtttt tcagagattt accacccagt taatggttag aaatattctg |
| 1801 | tggcaatgaa gaaaacacta ggggaaataa aaaataatat aaagccatat gagcttgtat |
| 1861 | tgaaatttgt tgcacttata ttgaaatgtg atcatggctc acttcagcct caacttacta |
| 1921 | agctcaagag gttctctcac ctcagccccc caagtagctg ggaccatagg tgcatgtcac |
| 1981 | catgtccaac taatttttta ttttttgtag tgatgagatc tcattgtgtt ctccatgctg |
| 2041 | atttcaaact cctgggctca aacaatcctc ccattttagc atcccaaagg gatgagatta |
| 2101 | caggtatgta ccaccataac tttacaaaat gagattttta tataagaatg attcaaatgt |
| 2161 | tcagggatga aagagtcact aacataaaag aagaatggga tgaggtgaga aggatgaata |
| 2221 | caaaaataat tagatattct tgaaatcaga aatgtgctcc ctaattatat gaaatgttgt |
| 2281 | ttgattacat aaaataaagt ggaaatgaat gattgactga acagcccaca agaagaatca |
| 2341 | cttaatgctc tgaaaattac cagtaaactg attaaaatct aaaattgctt tctgttaaag |
| 2401 | ctttactgat tagtttttct tccaaagctc tcttgtttct agttgttttc ttggtcttaa |
| 2461 | ctacccatta tatgctttgt taaagtgttt atgccctgat tcaatgtgat tatctcaatt |
| 2521 | tttatttcat tctgtcctaa ctcttgcaac ctgcatgtcc tctttattat tgatcaatcc |
| 2581 | aactgcaaag ttcaccttac ctgactaagg attattcatt aagttttact tgtttatctg |
| 2641 | acatttatta ttttgtctct ttgctagtca ctctgagcca tggtcatgat gacttaggat |
| 2701 | tctggatctc ttatgaataa caaatttatc cttaataaag tctctatact aaagaa |

UGT1A4 is a well-known UDP glucuronosyltransferase, see for example, Kerdpin O, et al., Drug Metabolism and Disposition. 2009 Sep. 1, 2009; 37(9):1948-55; Li D, et al., Journal of Biological Chemistry. 2007 Dec. 14, 2007; 282(50):36514-24; Kubota T, et al., Molecular Pharmacology. 2007 Oct. 1, 2007; 72(4):1054-62; Finel M, et al., Journal of Pharmacology and Experimental Therapeutics. 2005 Dec. 1, 2005; 315(3):1143-9; and Ritter J K, et al., Journal of Biological Chemistry. 1991 Jan. 15, 1991; 266(2):1043-7.

Precursor and mature human UGT1A4 amino acid sequences are shown herein as SEQ ID No. 3 and SEQ ID No. 4, respectively. A nucleic acid sequence encoding the UGT1A4 proteins of SEQ ID No. 3 and 4 is shown herein as SEQ ID No. 5. In humans the UGT1A4 gene is on chromosome: 2; location: 2q37.

Precursor UGT1A4 amino acid sequence SEQ ID NO. 3 (amino acid 24 P underlined and in bold; amino acid 48 L underlined and in bold):

| | |
|---|---|
| 1 | MARGLQVPLP RLATGLLLLL SVQPWAESGK VLVVPTDGSP WLSMREALRE LHARGHQAVV |
| 61 | LTPEVNMHIK EEKFFTLTAY AVPWTQKEFD RVTLGYTQGF FETEHLLKRY SRSMAIMNNV |
| 121 | SLALHRCCVE LLHNEALIRH LNATSFDVVL TDPVNLCGAV LAKYLSIPAV FFWRYIPCDL |
| 181 | DFKGTQCPNP SSYIPKLLTT NSDHMTFLQR VKNMLYPLAL SYICHTFSAP YASLASELFQ |
| 241 | REVSVVDLVS YASVWLFRGD FVMDYPRPIM PNMVFIGGIN CANGKPLSQE FEAYINASGE |
| 301 | HGIVVFSLGS MVSEIPEKKA MAIADALGKI PQTVLWRYTG TRPSNLANNT ILVKWLPQND |
| 361 | LLGHPMTRAF ITHAGSHGVY ESICNGVPMV MMPLFGDQMD NAKRMETKGA GVTLNVLEMT |
| 421 | SEDLENALKA VINDKSYKEN IMRLSSLHKD RPVEPLDLAV FWVEFVMRHK GAPHLRPAAH |
| 481 | DLTWYQYHSL DVIGFLLAVV LTVAFITFKC CAYGYRKCLG KKGRVKKAHK SKTH |

UGT1A4 mature amino acid sequence SEQ ID NO. 4 (amino acid “24” P underlined and in bold; amino acid “48” L underlined and in bold):

| | |
|---|---|
| 1 | GLQVPLPRLA TGLLLLLSVQ PWAESGKVLV VPTDGSPWLS MREALRELHA RGHQAVVLTP |
| 61 | EVNMHIKEEK FFTLTAYAVP WTQKEFDRVT LGYTQGFFET EHLLKRYSRS MAIMNNVSLA |
| 121 | LHRCCVELLH NEALIRHLNA TSFDVVLTDP VNLCGAVLAK YLSIPAVFFW RYIPCDLDFK |
| 181 | GTQCPNPSSY IPKLLTTNSD HMTFLQRVKN MLYPLALSYI CHTFSAPYAS LASELFQREV |
| 241 | SVVDLVSYAS VWLFRGDFVM DYPRPIMPNM VFIGGINCAN GKPLSQEFEA YINASGEHGI |
| 301 | VVFSLGSMVS EIPEKKAMAI ADALGKIPQT VLWRYTGTRP SNLANNTILV KWLPQNDLLG |
| 361 | HPMTRAFITH AGSHGVYESI CNGVPMVMMP LFGDQMDNAK RMETKGAGVT LNVLEMTSED |
| 421 | LENALKAVIN DKSYKENIMR LSSLHKDRPV EPLDLAVFWV EFVMRHKGAP HLRPAAHDLT |
| 481 | WYQYHSLDVI GFLLAVVLTV AFITFKCCAY GYRKCLGKKG RVKKAHKSKT H |

UGT1A4 Nucleic acid sequence SEQ ID NO. 5 (START codon and codon 48 underlined and in bold):

| | |
|---|---|
| 1 | acagtcagct gtcggtggct tctgctgaga tggccagagg actccaggtt cccctgccgc |
| 61 | ggctggccac aggactgctg ctcctcctca gtgtccagcc ctgggctgag agtggaaagg |
| 121 | tgttggtggt gcccactgat ggcagcccct ggctcagcat gcgggaggcc ttgcgggagc |
| 181 | tccatgccag aggccaccag gcggtggtcc tcaccccaga ggtgaatatg cacatcaaag |
| 241 | aagagaaatt tttcaccctg acagcctatg ctgttccatg gacccagaag gaatttgatc |
| 301 | gcgttacgct gggctacact caagggttct ttgaaacaga acatcttctg aagagatatt |
| 361 | ctagaagtat ggcaattatg aacaatgtat ctttggccct tcataggtgt tgtgtggagc |
| 421 | tactgcataa tgaggccctg atcaggcacc tgaatgctac ttcctttgat gtggttttaa |
| 481 | cagaccccgt taacctctgt ggggcggtgc tggctaagta cctgtcgatt cctgctgtgt |
| 541 | ttttttggag gtacattcca tgtgacttag actttaaggg cacacagtgt ccaaatcctt |
| 601 | cctcctatat tcctaagtta ctaacgacca attcagacca catgacattc ctgcaaaggg |
| 661 | tcaagaacat gctctaccct ctggccctgt cctacatttg ccatactttt tctgcccctt |
| 721 | atgcaagtct tgcctctgag ctttttcaga gagaggtgtc agtggtggat cttgtcagct |
| 781 | atgcatccgt gtggctgttc cgaggggact ttgtgatgga ctaccccagg ccgatcatgc |
| 841 | ccaacatggt cttcattggg ggcatcaact gtgccaacgg gaagccacta tctcaggaat |
| 901 | ttgaagccta cattaatgct tctggagaac atggaattgt ggttttctct ttgggatcaa |
| 961 | tggtctcaga aattccagag aagaaagcta tggcaattgc tgatgctttg ggcaaaatcc |
| 1021 | ctcagacagt cctgtggcgg tacactggaa cccgaccatc gaatcttgcg aacaacacga |
| 1081 | tacttgttaa gtggctaccc caaaacgatc tgcttggtca cccgatgacc cgtgccttta |
| 1141 | tcacccatgc tggttcccat ggtgtttatg aaagcatatg caatggcgtt cccatggtga |
| 1201 | tgatgccctt gtttggtgat cagatggaca atgcaaagcg catggagact aagggagctg |
| 1261 | gagtgaccct gaatgttctg gaaatgactt ctgaagattt agaaaatgct ctaaaagcag |
| 1321 | tcatcaatga caaaagttac aaggagaaca tcatgcgcct ctccagcctt cacaaggacc |
| 1381 | gcccggtgga gccgctggac ctggccgtgt tctgggtgga gtttgtgatg aggcacaagg |
| 1441 | gcgcgccaca cctgcgcccc gcagcccacg acctcacctg gtaccagtac cattccttgg |
| 1501 | acgtgattgg tttcctcttg gccgtcgtgc tgacagtggc cttcatcacc tttaaatgtt |
| 1561 | gtgcttatgg ctaccggaaa tgcttgggga aaaaagggcg agttaagaaa gcccacaaat |
| 1621 | ccaagaccca ttgagaagtg ggtgggaaat aaggtaaaat tttgaaccat tccctagtca |
| 1681 | tttccaaact tgaaaacaga atcagtgtta aattcatttt attcttatta aggaaatact |
| 1741 | ttgcataaat taatcagccc cagagtgctt taaaaaattc tcttaaataa aaataataga |
| 1801 | ctcgctagtc actaaaaata tttgaatatg tatcgtgccc cctctggtgt ctttgatcag |
| 1861 | gatgacatgt gccatttttc agaggacgtg cagacaggct ggcattctag attacttttc |
| 1921 | ttactctgaa acatggcctg tttgggagtg cgggattcaa aggtggtccc acggctgccc |
| 1981 | cttatgtcaa tggcagtttt aatcttatct tttggcttct gcagatggtt gcaattgatc |
| 2041 | cttaaccaat aatggtcagt cctcatctct gtcgtgcttc ataggtgcca ccttgtgtgt |
| 2101 | ttaaagaagg gaagctttgt acctttagag tgtaggtgaa atgaatgaat ggcttggagt |
| 2161 | gcactgagaa cagcatatga tttcttgctt tggggaaaaa gaatgatgct atgaaattgg |
| 2221 | tgggtggtgt atttgagaag ataatcattg cttatgtcaa atggagctga atttgataaa |
| 2281 | aacccaaaat acagctatga agtgctgggc aagtttactt tttttctgat gtttcctaca |
| 2341 | actaaaaata aattaataaa tttatataaa ttct |

Primers that can be used to determine the identity of amino acid 67 of UGT2B10 further include: sense (forward) primer 5′-AAGGATGGCTCTGAAATGGACTA-3′ SEQ ID NO. 10 and antisense (reverse) primer 5′-ATGAGTAGCCAGGACTGAAGCTGT-3′ SEQ ID NO. 11 (see Chen G, et al., Pharmacogenetics and Genomics 2008; 18(3):181-91; and Chen G, et al., Drug Metab Dispos. 2008 May 1, 2008; 36(5):824-30); sense primer 5′-ATGGCTCTGAAATGGACTACAGTT-3′ SEQ ID NO. 12 and antisense primer 5′-CCAGCTTCAAATCTCAGATATAAC-3′ SEQ ID NO. 13 (see Thou D, et al., Drug Metabolism and Disposition, May 2010; 38(5):863-70); and sense primer 5′-GGC TCT GAA ATG GAC TA-3′ SEQ ID NO. 14 and 5′-TGA TGA TAA ATA GCA CG-3′ SEQ ID NO. 15 (see Izukawa T, et al., Drug Metabolism and Disposition, 2009 August 2009; 37(8):1759-68)

Primers that can be used to determine the identity of amino acids 24 and/or 48 of UGT1A4 further include: sense (forward) primer 5′-ACAGTCAGCTGTCGGTGG-3′ SEQ ID NO. 16 and antisense (reverse) primer 5′-GCACTCTGGGGCTGATTAAT-3′ SEQ ID NO. 17 (see Fujiwara R, et al., Drug Metabolism and Disposition. 2007 May 1, 2007; 35(5):747-57); sense primer 5′-GCTTCTGCTGAGATGGCCAG-3′ SEQ ID NO. 18 and antisense primer 5′-GCACTCTGGGGCTGATTAAT-3′ SEQ ID NO. 17 (see Fujiwara R, et al., Drug Metabolism and Disposition. 2007 May 1, 2007; 35(5):747-57); sense primer 5′-GAATTCATGGCCAGAGGACTCCAG-3′ SEQ ID NO. 19 and antisense primer 5′-CTCGAGTCAATGGGTCTTGGATTTG-3′ SEQ ID NO. 20 (see Qian M, et al., Biochemical and Biophysical Research Communications. 2004; 319(2):386-92); sense primer 5′-ACAGTCAGCTGTCGGTGGC-3′ SEQ ID NO. 21 and antisense primer 5′-ATTTTACCTTATTTCCCACCC-3′ SEQ ID NO. 22 (see Sun D, et al., Breast Cancer Res. 2006; 8(4):R50); sense primer 5′-TTAACAAAGTAGAAGGCAGTG-3′ SEQ ID NO. 23 and antisense primer 5′-TGAAAACTTGAAATACACTAGGC-3′ SEQ ID NO. 24 (see Saeki M, et al., Drug Metabolism and Pharmacokinetics. 2005; 20(2):144-51); sense primer 5′-GTTGGGCCCATAACGAAAGGCAGTT-3′ SEQ ID NO.25 and antisense primer 5′-TGGAACATTGATTGGATGAAGGCACC-3′ SEQ ID NO. 26 (see Ehmer U, et al., Hepatology. 2004; 39(4):970-7); sense primer 5′-GCCCATAACGAAAGGCAGT-3′ SEQ ID NO. 27 and antisense primer 5′-CCTCAACAGTTTATCTGTTATAAAGA-3′ SEQ ID NO. 28 (see Wiener D, et al., Cancer Res. 2004 Feb. 1; 64(3):1190-6); sense primer 5″-GAAGGAATTTGATCGCGTTAC-3′ SEQ ID NO. 29 and antisense primer 5′-CCAATGAAGACCATGTTGGGC-3′ SEQ ID NO. 30 see Strassburg C P, et al., Molecular Pharmacology. 1997 Aug. 1, 1997; 52(2):212-20; and others described in Benoit-Biancamano M O A J, et al., Pharmacogenetics and Genomics. 2009; 19(12):945-54; and Kurkela M, et al., Journal of Biological Chemistry. 2003 Feb. 7, 2003; 278(6):3536-44.

### REFERENCES

Blevins-Primeau A S, Sun D, Chen G, Sharma A K, Gallagher C J, Amin S, et al. Functional significance of UDP-glucuronosyltransferase variants in the metabolism of active tamoxifen metabolites. Cancer Res. 2009 Mar. 1, 2009; 69(5):1892-900.
Wiener D, Doerge D R, Fang J L, Upadhyaya P, Lazarus P. Characterization of N-glucuronidation of the lung carcinogen 4-(methylnitrosamino)-1-(3-pyridyl)-1-butanol (NNAL) in human liver: importance of UDP-glucuronosyltransferase 1A4. Drug Metab Dispos. 2004 January; 32(1):72-9.
Coughtrie M W, Burchell B, Bend J R. A general assay for UDPglucuronosyltransferase activity using polar amino-cyano stationary phase HPLC and UDP[U-14C]glucuronic acid. Anal Biochem. 1986 November; 159(1):198-205.
Chen G, Dellinger R W, Gallagher C J, Sun D, Lazarus P. Identification of a prevalent functional missense polymorphism in the UGT2B10 gene and its association with UGT2B10 inactivation against tobacco-specific nitrosamines. Pharmacogenetics and Genomics 2008; 18(3):181-91.
Chen G, Dellinger R W, Sun D, Spratt T E, Lazarus P. Glucuronidation of tobacco-specific nitrosamines by UGT2B10. Drug Metab Dispos. 2008 May 1, 2008; 36(5):824-30.
Ren Q, Murphy S E, Zheng Z, Lazarus P. O-Glucuronidation of the lung carcinogen 4-(methylnitrosamino)-1-(3-pyridyl)-1-butanol (NNAL) by human UDP-glucuronosyltransferases 2B7 and 1A9. Drug Metab Dispos. 2000 November; 28(11):1352-60.
Dellinger R W, Fang J L, Chen G, Weinberg R, Lazarus P. Importance of UDP-glucuronosyltransferase 1A10 (UGT1A10) in the detoxification of polycyclic aromatic hydrocarbons: decreased glucuronidative activity of the UGT1A10 139Lys isoform. Drug Metabolism and Disposition. 2006 June 2006; 34(6):943-9.
Sun D, Chen G, Dellinger R W, Duncan K, Fang J L, Lazarus P. Characterization of tamoxifen and 4-hydroxytamoxifen glucuronidation by human UGT1A4 variants. Breast Cancer Res. 2006; 8(4):R50.
Lazarus P, Blevins Primeau A S, Zheng Y, Sun D. Potential role of UGT pharmacogenetics in cancer treatment and prevention: Focus on tamoxifen. Ann N Y Acad Sci. 2009 February; 1155:99-111.
Chen G, Blevins-Primeau A S, Dellinger R W, Muscat J E, Lazarus P. Glucuronidation of Nicotine and Cotinine by UGT2B10: Loss of Function by the UGT2B10 Codon 67 (Asp>Tyr) Polymorphism. Cancer Research. 2007 Oct. 1, 2007; 67(19):9024-9.
Gallagher C J, Muscat J E, Hicks A N, Zheng Y, Dyer A-M, Chase G A, et al. The UDP-Glucuronosyltransferase 2B17 Gene Deletion Polymorphism: Sex-Specific Association with Urinary 4-(Methylnitrosamino)-1-(3-Pyridyl)-1-Butanol Glucuronidation Phenotype and Risk for Lung Cancer. Cancer Epidemiol Biomarkers Prey. 2007 Apr. 1, 2007; 16(4):823-8.
Kassahun K, Mattiuz E, Nyhart E, Jr., Obermeyer B, Gillespie T, Murphy A, et al. Disposition and biotransformation of the antipsychotic agent olanzapine in humans. Drug Metab Dispos. 1997 Jan. 1, 1997; 25(1):81-93.
Devane C L, Markowitz J S. Antipsychotics. 1st ed. Ambler: Lippencott Williams & Wilkins; 2000.
Calligaro D O, Fairhurst J, Hotten T M, Moore N A, Tupper D E. The synthesis and biological activity of some known and putative metabolites of the atypical antipsychotic agent olanzapine. Bioorganic and Medicinal Chemistry Letters. 1997; 7(1):25-30.
Kassahun K, Mattiuz E, Franklin R, Gillespie T. Olanzapine 10-N-glucuronide. A tertiary N-glucuronide unique to humans Drug Metab Dispos. 1998; 26(9):848-55.
Erichsen T J, Ehmer U, Kalthoff S, Lankisch T O, Müller T M, Munzel P A, et al. Genetic variability of aryl hydrocarbon receptor (AhR)-mediated regulation of the human UDP glucuronosyltransferase (UGT) 1A4 gene. Toxicology and Applied Pharmacology. 2008; 230(2):252-60.
Wiener D, Fang J L, Dossett N, Lazarus P. Correlation between UDP-glucuronosyltransferase genotypes and 4-(methylnitrosamino)-1-(3-pyridyl)-1-butanone glucuronidation phenotype in human liver microsomes. Cancer Res. 2004 Feb. 1; 64(3):1190-6.
Linnet K. Glucuronidation of olanzapine by cDNA-expressed human UDP-glucuronosyltransferases and human liver microsomes. Hum Psychopharmacol. 2002 July; 17(5):233-8.
Rowland A, Elliot D J, Williams J A, Mackenzie P I, Dickinson R G, Miners J O. In Vitro Characterization of Lamotrigine N2-glucuronidation and the lamotrigine-valproic acid interaction. Drug Metabolism and Disposition. 2006 Jun. 1, 2006; 34(6):1055-62.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for aiding in determining therapeutic efficacy of olanzapine in a subject, comprising:
obtaining a sample comprising genomic DNA or mRNA encoding UDP-glucuronosyltransferase 2B10 and/or UDP-glucuronosyltransferase 1A4 from the subject;
identifying the amino acid encoded by the genomic DNA or mRNA at amino acid position 67 of UDP-glucuronosyltransferase 2B10, wherein Tyr at position 67 of the UDP-glucuronosyltransferase 2B10 is correlated with decreased glucuronidation of olanzapine compared to Asp at position 67 and/or identifying the amino acid encoded by the genomic DNA or mRNA at amino acid position 24 and/or 48 of UDP-glucuronosyltransferase 1A4, wherein P24T variant UDP-glucuronosyltransferase 1A4 is correlated with decreased glucuronidation of olanzapine compared to wild-type UDP-glucuronosyltransferase 1A4 and wherein L48V variant UDP-glucuronosyltransferase 1A4 is correlated with increased glucuronidation of olanzapine compared to wild-type UDP-glucuronosyltransferase 1A4.

2. The method of claim 1 wherein the identifying comprises polymerase chain reaction.

3. The method of claim 1, wherein the subject is human.

4. A method for aiding in determining therapeutic efficacy of olanzapine in a subject, comprising:
obtaining a sample comprising UDP-glucuronosyltransferase 2B10 and/or UDP-glucuronosyltransferase 1A4 from the subject; and
identifying the UDP-glucuronosyltransferase 2B10 as wild-type or D67Y variant, wherein D67Y variant UDP-glucuronosyltransferase 2B10 is correlated with decreased glucuronidation of olanzapine compared to wild-type UDP-glucuronosyltransferase 2B10 and/or identifying the UDP-glucuronosyltransferase 1A4 as wild-type, P24T variant or L48V variant, wherein P24T variant UDP-glucuronosyltransferase 1A4 is correlated with decreased glucuronidation of olanzapine compared to wild-type UDP-glucuronosyltransferase 1A4, wherein L48V variant is correlated with increased glucuronidation of olanzapine compared to wild-type UDP-glucuronosyltransferase 1A4.

5. The method of claim 4, wherein the subject is human.

6. The method of claim 4, wherein the identifying comprises immunoassay, mass spectrometry and/or chromatography.

7. A method for aiding in determining therapeutic efficacy of olanzapine in a subject, comprising: measuring expression and/or function of UDP-glucuronosyltransferase 1A4 and/or UDP-glucuronosyltransferase 2B10 in a subject sample, wherein expression and/or function of UDP-glucuronosyltransferase 2B10 and UDP-glucuronosyltransferase 1A4 is correlated with therapeutic efficacy of olanzapine in the subject.

8. A kit for aiding in determining therapeutic efficacy of olanzapine in a subject, comprising:
at least one reagent for detection of a UDP-glucuronosyltransferase 2B10 nucleic acid or protein and/or at least one reagent for detection of a UDP-glucuronosyltransferase 1A4 nucleic acid or protein.

9. The kit of claim 8 wherein the reagent is a nucleic acid probe or primer for detection of a D67Y variant UDP-glucuronosyltransferase 2B10 nucleic acid.

10. The kit of claim 8 wherein the reagent is a nucleic acid probe or primer for detection of a P24T variant UDP-glucuronosyltransferase 1A4 nucleic acid.

11. The kit of claim 8 wherein the reagent is a nucleic acid probe or primer for detection of a L48V variant UDP-glucuronosyltransferase 1A4 nucleic acid.

12. The kit of claim 8 wherein the reagent is an antibody or aptamer.


SEQUENCE LISTING (30 sequences)

SEQ ID NO: 1 - protein, 528 aa, Homo sapiens
MALKWTTVLLIQLSFYFSSGSCGKVLVWAAEYSLWMNMKTILKELVQRGHEVTVLASSASILFDPNDSSTLKLEVYPTSLTKTEFENIIMQLVKRLSEIQKDTFWLPFSQEQEILWAINDIIRNFCKDVVSNKKLMKKLQESRFDIVFADAYLPCGELLAELFNIPFVYSHSFSPGYSFERHSGGFIFPPSYVPVVMSKLSDQMTFMERVKNMLYVLYFDFWFQIFNMKKWDQFYSEVLGRPTTLSETMRKADIWLMRNSWNFKFPHPFLPNVDFVGGLHCKPAKPLPKEMEEFVQSSGENGVVVFSLGSMVSNMTEERANVIATALAKIPQKVLWRFDGNKPDALGLNTRLYKWIPQNDLLGHPKTRAFITHGGANGIYEAIYHGIPMVGIPLFFDQPDNIAHMKAKGAAVRVDFNTMSSTDLLNALKTVINDPSYKENIMKLSRIQHDQPVKPLDRAVFWIEFVMRHKGAKHLRVAAHNLTWFQYHSLDVIGFLLACVATVLFIITKCCLFCFWKFARKGKKGKRD

SEQ ID NO: 2 - DNA, 2756 bp, Homo sapiens
aaaagaattatcacattgcacaaggatggctctgaaatggactacagttctgctgatacaactcagtttttactttagctctgggagttgtggaaaggtgctggtatgggccgcagaatacagcctttggatgaatatgaagacaatcctgaaagaacttgttcagagaggtcatgaggtgactgtactggcatcttcagcttccattctttttgatcccaacgactcatccactcttaaacttgaagtttatcctacatctttaactaaaactgaatttgagaatatcatcatgcaattggttaagagattgtcagaaattcaaaaagatacattttggttacctttttcacaagaacaagaaatcctgtgggcaattaatgacataattagaaacttctgtaaagatgtagtttcaaataagaaacttatgaaaaaactacaagagtcaagatttgacatcgtttttgcagatgcttatttaccctgtggtgagctgctggctgagctatttaacataccctttgtgtacagtcacagcttcagtcctggctactcatttgaaaggcacagtggaggatttattttccctccttcctacgtacctgttgttatgtcaaaattaagtgatcaaatgactttcatggagagggtaaaaaatatgctctatgtgctttattttgacttttggttccaaatatttaatatgaagaagtgggatcagttttacagtgaagttttaggaagacccactacattatctgagacaatgaggaaagctgacatatggcttatgcgaaactcctggaattttaaatttcctcatccattcttaccaaatgttgattttgttggaggactccactgcaaacctgccaaacccctacctaaggaaatggaggagtttgtacagagctctggagaaaatggtgttgtggtgttttctctggggtcaatggtcagtaacatgacagaagaaagggccaacgtaattgcaacagcccttgccaagatcccacaaaaggttctttggagatttgatgggaataaaccagatgccttaggtctcaatactcgactgtacaagtggataccccagaatgaccttctaggtcatccaaaaaccagagcttttataactcatggtggagccaatggcatctatgaggcaatctaccatgggatccctatggtgggcattccattgttttttgatcaacctgataatattgctcacatgaaggccaagggagcagctgttagagtggacttcaacacaatgtcgagtacagacctgctgaatgcactgaagacagtaattaatgatccttcatataaagagaatattatgaaattatcaagaattcaacatgatcaaccagtgaagcccctggatcgagcagtcttctggattgaatttgtcatgcgccacaaaggagccaaacatcttcgagttgcagcccacaacctcacctggttccagtaccactctttggatgtgattgggttcctgctggcttgtgtggcaaccgtgctatttatcatcacaaagtgttgtctgttttgtttctggaagtttgctagaaaaggaaagaagggaaaaagggattagttatatctgagatttgaagctggaaaacctgatagataggaatacttcagttgattccagcaataaatattgtgatgcaagatttctttcttcctgtgacaaaaaaaaatcctttcgaagtctaccttgtcaagtaaaaatttgtttttcagagatttaccacccagttaatggttagaaatattctgtggcaatgaagaaaacactaggggaaataaaaaataatataaagccatatgagcttgtattgaaatttgttgcacttatattgaaatgtgatcatggctcacttcagcctcaacttactaagctcaagaggttctctcacctcagccccccaagtagctgggaccataggtgcatgtcaccatgtccaactaattttttattttttgtagtgatgagatctcattgtgttctccatgctgatttcaaactcctgggctcaaacaatcctcccattttagcatcccaaagggatgagattacaggtatgtaccaccataactttacaaaatgagatttttatataagaatgattcaaatgttcagggatgaaagagtcactaacataaaagaagaatgggatgaggtgagaaggatgaatacaaaaataattagatattcttgaaatcagaaatgtgctccctaattatatgaaatgttgtttgattacataaaataaagtggaaatgaatgattgactgaacagcccacaagaagaatcacttaatgctctgaaaattaccagtaaactgattaaaatctaaaattgctttctgttaaagctttactgattagtttttcttccaaagctctcttgtttctagttgttttcttggtcttaactacccattatatgctttgttaaagtgtttatgccctgattcaatgtgattatctcaatttttatttcattctgtcctaactcttgcaacctgcatgtcctctttattattgatcaatccaactgcaaagttcaccttacctgactaaggattattcattaagttttacttgtttatctgacatttattattttgtctctttgctagtcactctgagccatggtcatgatgacttaggattctggatctcttatgaataacaaatttatccttaataaagtctctatactaaagaa

SEQ ID NO: 3 - protein, 534 aa, Homo sapiens
MARGLQVPLPRLATGLLLLLSVQPWAESGKVLVVPTDGSPWLSMREALRELHARGHQAVVLTPEVNMHIKEEKFFTLTAYAVPWTQKEFDRVTLGYTQGFFETEHLLKRYSRSMAIMNNVSLALHRCCVELLHNEALIRHLNATSFDVVLTDPVNLCGAVLAKYLSIPAVFFWRYIPCDLDFKGTQCPNPSSYIPKLLTTNSDHMTFLQRVKNMLYPLALSYICHTFSAPYASLASELFQREVSVVDLVSYASVWLFRGDFVMDYPRPIMPNMVFIGGINCANGKPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 4 - protein, 531 aa, Homo sapiens
GLQVPLPRLATGLLLLLSVQPWAESGKVLVVPTDGSPWLSMREALRELHARGHQAVVLTPEVNMHIKEEKFFTLTAYAVPWTQKEFDRVTLGYTQGFFETEHLLKRYSRSMAIMNNVSLALHRCCVELLHNEALIRHLNATSFDVVLTDPVNLCGAVLAKYLSIPAVFFWRYIPCDLDFKGTQCPNPSSYIPKLLTTNSDHMTFLQRVKNMLYPLALSYICHTFSAPYASLASELFQREVSVVDLVSYASVWLFRGDFVMDYPRPIMPNMVFIGGINCANGKPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 5 - DNA, 2374 bp, Homo sapiens
acagtcagctgtcggtggcttctgctgagatggccagaggactccaggttcccctgccgcggctggccacaggactgctgctcctcctcagtgtccagccctgggctgagagtggaaaggtgttggtggtgcccactgatggcagcccctggctcagcatgcgggaggccttgcgggagctccatgccagaggccaccaggcggtggtcctcaccccagaggtgaatatgcacatcaaagaagagaaatttttcaccctgacagcctatgctgttccatggacccagaaggaatttgatcgcgttacgctgggctacactcaagggttctttgaaacagaacatcttctgaagagatattctagaagtatggcaattatgaacaatgtatctttggcccttcataggtgttgtgtggagctactgcataatgaggccctgatcaggcacctgaatgctacttcctttgatgtggttttaacagaccccgttaacctctgtggggcggtgctggctaagtacctgtcgattcctgctgtgtttttttggaggtacattccatgtgacttagactttaagggcacacagtgtccaaatccttcctcctatattcctaagttactaacgaccaattcagaccacatgacattcctgcaaagggtcaagaacatgctctaccctctggccctgtcctacatttgccatactttttctgccccttatgcaagtcttgcctctgagctttttcagagagaggtgtcagtggtggatcttgtcagctatgcatccgtgtggctgttccgaggggactttgtgatggactaccccaggccgatcatgcccaacatggtcttcattgggggcatcaactgtgccaacgggaagccactatctcaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctctggtgtctttgatcaggatgacatgtgccatttttcagaggacgtgcagacaggctggcattctagattacttttcttactctgaaacatggcctgtttgggagtgcgggattcaaaggtggtcccacggctgcccctactgcaaatggcagttttaatcttatcttttggcttctgcagatggttgcaattgatccttaaccaataatggtcagtcctcatctctgtcgtgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattct

SEQ ID NO: 6 - DNA, 21 bp, artificial
  UGT1A4 forward primer
ggcttctgctgagatggccag

SEQ ID NO: 7 - DNA, 20 bp, artificial
  UGT1A4 reverse primer
ccttgagtgtagcccagcgt

SEQ ID NO: 8 - DNA, 23 bp, artificial
  UGT2B10 forward primer
aaggatggctctgaaatggacta

SEQ ID NO: 9 - DNA, 24 bp, artificial
  UGT2B10 reverse primer
atgagtagccaggactgaagctgt

SEQ ID NO: 10 - DNA, 23 bp, artificial
  UGT2B10 forward primer
aaggatggctctgaaatggacta

SEQ ID NO: 11 - DNA, 24 bp, artificial
  UGT2B10 reverse primer
atgagtagccaggactgaagctgt

SEQ ID NO: 12 - DNA, 24 bp, artificial
  UGT2B10 forward primer
atggctctgaaatggactacagtt

SEQ ID NO: 13 - DNA, 24 bp, artificial
  UGT2B10 reverse primer
ccagcttcaaatctcagatataac

SEQ ID NO: 14 - DNA, 17 bp, artificial
  UGT2B10 forward primer
ggctctgaaatggacta

SEQ ID NO: 15 - DNA, 17 bp, artificial
  UGT2B10 reverse primer
tgatgataaatagcacg

SEQ ID NO: 16 - DNA, 18 bp, artificial
  UGT1A4 forward primer
acagtcagctgtcggtgg

SEQ ID NO: 17 - DNA, 20 bp, artificial
  UGT1A4 reverse primer
gcactctggggctgattaat

SEQ ID NO: 18 - DNA, 20 bp, artificial
  UGT1A4 forward primer
gcttctgctgagatggccag

SEQ ID NO: 19 - DNA, 24 bp, artificial
  UGT1A4 forward primer
gaattcatggccagaggactccag

SEQ ID NO: 20 - DNA, 25 bp, artificial
  UGT1A4 reverse primer
ctcgagtcaatgggtcttggatttg

SEQ ID NO: 21 - DNA, 19 bp, artificial
  UGT1A4 forward primer
acagtcagctgtcggtggc

SEQ ID NO: 22 - DNA, 21 bp, artificial
  UGT1A4 reverse primer
attttaccttatttcccaccc

SEQ ID NO: 23 - DNA, 21 bp, artificial
  UGT1A4 forward primer
ttaacaaagtagaaggcagtg

SEQ ID NO: 24 - DNA, 23 bp, artificial
  UGT1A4 reverse primer
tgaaaacttgaaatacactaggc

SEQ ID NO: 25 - DNA, 25 bp, artificial
  UGT1A4 forward primer
gttgggcccataacgaaaggcagtt

SEQ ID NO: 26 - DNA, 26 bp, artificial
  UGT1A4 reverse primer
tggaacattgattggatgaaggcacc

SEQ ID NO: 27 - DNA, 19 bp, artificial
  UGT1A4 forward primer
gcccataacgaaaggcagt

SEQ ID NO: 28 - DNA, 26 bp, artificial
  UGT1A4 reverse primer
cctcaacagtttatctgttataaaga

SEQ ID NO: 29 - DNA, 21 bp, artificial
  UGT1A4 forward primer
gaaggaatttgatcgcgttac

SEQ ID NO: 30 - DNA, 21 bp, artificial
  UGT1A4 reverse primer
ccaatgaagaccatgttgggc